SEQUENCE LISTING (27 sequences)

SEQ ID NO: 1 - DNA, 30 bp, synthetic construct
  misc_feature 1..30: note = chemically synthesized
  source 1..30: mol_type = other DNA
gggggggggggacgatcgtcgggggggggg

SEQ ID NO: 2 - DNA, 10 bp, synthetic construct
  misc_feature 1..10: note = chemically synthesized
  source 1..10: mol_type = other DNA
gacgatcgtc

SEQ ID NO: 3 - DNA, 31 bp, synthetic construct
  misc_feature 1..31: note = chemically synthesized
  source 1..31: mol_type = other DNA
gggggggggggacgatcgtcggggggggggg

SEQ ID NO: 4 - DNA, 33 bp, synthetic construct
  misc_feature 1..33: note = chemically synthesized
  source 1..33: mol_type = other DNA
gggggggggggggacgatcgtcggggggggggg

SEQ ID NO: 5 - DNA, 22 bp, synthetic construct
  misc_feature 1..22: note = chemically synthesized
  source 1..22: mol_type = other DNA
gggggggacgatcgtcgggggg

SEQ ID NO: 6 - DNA, 24 bp, synthetic construct
  misc_feature 1..24: note = chemically synthesized
  source 1..24: mol_type = other DNA
ggggggggacgatcgtcggggggg

SEQ ID NO: 7 - DNA, 26 bp, synthetic construct
  misc_feature 1..26: note = chemically synthesized
  source 1..26: mol_type = other DNA
gggggggggacgatcgtcgggggggg

SEQ ID NO: 8 - DNA, 28 bp, synthetic construct
  misc_feature 1..28: note = chemically synthesized
  source 1..28: mol_type = other DNA
ggggggggggacgatcgtcggggggggg

SEQ ID NO: 9 - DNA, 32 bp, synthetic construct
  misc_feature 1..32: note = chemically synthesized
  source 1..32: mol_type = other DNA
ggggggggggggacgatcgtcggggggggggg

SEQ ID NO: 10 - protein, 132 aa, Bacteriophage Qb
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 11 - protein, 329 aa, bacteriophage Qb
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 12 - protein, 129 aa, bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 13 - protein, 130 aa, bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 14 - protein, 130 aa, bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 15 - protein, 132 aa, bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 16 - protein, 329 aa, bacteriophage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 17 - protein, 130 aa, bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 18 - protein, 133 aa, bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 19 - protein, 133 aa, bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 20 - protein, 330 aa, bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 21 - protein, 129 aa, bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 22 - protein, 128 aa, bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 23 - protein, 131 aa, bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 24 - DNA, 26 bp, synthetic construct
  misc_feature 1..26: note = G6-10
  source 1..26: mol_type = other DNA
gggggggacgatcgtcgggggggggg

SEQ ID NO: 25 - DNA, 27 bp, synthetic construct
  misc_feature 1..27: note = G7-10
  source 1..27: mol_type = other DNA
ggggggggacgatcgtcgggggggggg

SEQ ID NO: 26 - DNA, 28 bp, synthetic construct
  misc_feature 1..28: note = G8-10
  source 1..28: mol_type = other DNA
gggggggggacgatcgtcgggggggggg

SEQ ID NO: 27 - DNA, 29 bp, synthetic construct
  misc_feature 1..29: note = G9-10
  source 1..29: mol_type = other DNA
ggggggggggacgatcgtcgggggggggg

(19) United States
(12) Patent Application Publication
(10) Pub. No.: US 2025/0019695 A1
(43) Pub. Date: Jan. 16, 2025

(54) PACKAGING OLIGONUCLEOTIDES INTO VIRUS-LIKE PARTICLES

(71) Applicant: CHECKMATE PHARMACEUTICALS, INC., Cambridge, MA (US)

(72) Inventors: Evan David WALTERS, Cambridge, MA (US); Frank HENNECKE, Cambridge, MA (US)

(21) Appl. No.: 18/780,329

(22) Filed: Jul. 22, 2024

Related U.S. Application Data
(62) Division of application No. 17/045,929, filed on Oct. 7, 2020, now Pat. No. 12,084,655.
(60) Provisional application No. 62/654,586, filed on Apr. 9, 2018.

(51) Int. Cl. C12N 15/11; C12N 7/00

(52) U.S. Cl. CPC C12N 15/111; C12N 7/00; C12N 2795/18123

(57) ABSTRACT

The present invention relates to processes for producing compositions comprising (i) a virus-like particle of an RNA bacteriophage, and (ii) aggregated oligonucleotides, wherein said aggregated oligonucleotides are packaged into said virus-like particle. The invention further provides processes for producing nucleotide compositions comprising aggregated oligonucleotides suitable for use in the aforementioned processes before. Moreover, the invention further provides nucleotide compositions comprising aggregated oligonucleotides. Furthermore, the invention further provides compositions comprising (i) a virus-like particle of an RNA bacteriophage, and (ii) aggregated oligonucleotides, wherein said aggregated oligonucleotides are packaged into said virus-like particle.

### RELATED APPLICATIONS

This application is a divisional of U.S. patent application Ser. No. 17/045,929, filed Oct. 7, 2020, now U.S. Pat. No. 12,084,655, which is a 35 U.S.C. § 371 filing of International Patent Application No. PCT/IB2019/052867, filed Apr. 8, 2019, which claims priority to U.S. Provisional Patent Application Ser. No. 62/654,586, filed Apr. 9, 2018, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Sep. 30, 2024, is named 756377_RGN9-008DIV_ST26.xml and is 31,936 bytes in size.

The present invention relates to processes for producing compositions comprising (i) a virus-like particle of an RNA bacteriophage, and (ii) aggregated oligonucleotides, wherein said aggregated oligonucleotides are packaged into said virus-like particle. The invention further provides processes for producing nucleotide compositions comprising aggregated oligonucleotides suitable for use in the aforementioned processes before. Moreover, the invention further provides nucleotide compositions comprising aggregated oligonucleotides. Furthermore, the invention further provides compositions comprising (i) a virus-like particle of an RNA bacteriophage, and (ii) aggregated oligonucleotides, wherein said aggregated oligonucleotides are packaged into said virus-like particle.

### RELATED ART

Virus-like particles of RNA bacteriophages packaged with oligonucleotides, in particular guanine (G)-rich oligonucleotides, are suggested as potent stimulators of the immune system. Such virus-like particles and oligonucleotides packaged therein and processes for their production have been described, for example, in WO2003/024481, WO2004/000351, WO2004/084940, WO2004/007538, WO2007/068747 and WO2007/144150, the entire disclosures of which are incorporated by reference herein. Typically, the processes are based on the disassembly of a recombinant virus-like particle of RNA bacteriophage, the purification of the coat protein of said virus-like particle and the reassembly of said coat protein in the presence of the oligonucleotides leading to the virus-like particles packaged with the oligonucleotides. Efficient and scalable processes for the production of recombinant virus-like particles of RNA bacteriophages are further disclosed, for example, in WO2005/117963, WO2006/125821 and WO2007/039552, which are incorporated herein by reference in its entirety.

Methods of oligonucleotide synthesis have been available for over thirty years with the synthesis through phosphoramidite chemistry being the most commonly used method thereof (Beaucage et al., Curr Protoc Nucleic Acid Chem 3.3.1-3.3.20 (2000), the entire disclosure of which is incorporated herein by reference). There have been many significant improvements to phosphoramidite synthesis to reduce synthesis time and generate higher yield of products. The synthesis of guanine (G)-rich oligonucleotides, in particular the ones with consecutive guanine residues, was always more challenging to achieve at large manufacturing scale, in particular at high purity and high yields, likely due to the poor accessibility of the 5′-hydroxyl group by the activated phosphoramidite in the coupling step. In particular, the support-bound protected G-rich oligomer can form secondary structures and have solubility issues after a certain length leading to impurities and synthesis failures most dominantly leading to oligonucleotide sequences with lower number of G residues or even higher G residues as desired. As a consequence, the purity of the commercially available guanine (G)-rich oligonucleotides has benefited significantly from said improvements made to phosphoramidite synthesis. Thus, while a purity of 60-80% for said guanine (G)-rich oligonucleotide had occasionally been accepted 10-20 years ago, a purity as high as 93%, 95%, or even as high as 97 or 99% in analogy to the non-guanine (G)-rich oligonucleotides, is nowadays generally feasible. Moreover, if said (G)-rich oligonucleotides are part of pharmaceutical drugs, the use of such higher purity (G)-rich oligonucleotides is required and requested for regulatory approval.

G-rich oligonucleotides, in particular those with poly (G) s at the 5′ and 3′ end and further comprising unmethylated CG dinucleotide motifs and a central palindrome, have the tendency to self-assemble to higher order secondary and tertiary structures via G-tetrad formation of their poly(G) motifs (Kerkmann, M. et al., J. Biol. Chem., (2005) 280(9), 8086-93, Bochman, ML et al. Nat Rev Genet., (2012) 13(11): 770-780; the entire disclosures of which are incorporated by reference herein). These G-quadruplexes can form via inter or intra molecular pathway and are very stable secondary structures. As a result, the size and shape and conformation of these quadruplexes can be quite variable depending on the reaction pathway.

WO2007/144150 describes a process for producing compositions comprising guanine (G)-rich oligonucleotides packaged into virus-like particles of an RNA bacteriophage, wherein the self-assembly of the coat protein of the RNA bacteriophage is performed in the presence of oligonucleotide aggregates which have been obtained by a disaggregation/aggregation process. The aggregation state of the oligonucleotide is characterized by the relative peak start time (PST) in size exclusion HPLC using the capsid of said RNA bacteriophage as standard and PST's of 50 to 110%, preferably of 80 to 95%, has been found to be optimal. It is indicated that such PSTs correspond to oligonucleotide aggregates having an apparent molecular weight in the range of the apparent molecular weight of the capsid of said RNA bacteriophage or slightly below. Despite its improvements over the known processes for producing compositions comprising guanine (G)-rich oligonucleotides packaged into virus-like particles of RNA bacteriophages, the present inventors have identified substantial disadvantages of this prior art process of WO2007/144150.

### SUMMARY OF THE INVENTION

In particular, it has been found by the present inventors that aggregated oligonucleotides prepared in accordance with the prior art process of WO2007/144150 and being in accordance with the size distribution as defined in WO2007/144150 showed substantial inconsistencies and large variations with respect to the specific size and conformation of the formed aggregated oligonucleotides, as determined by Dynamic Light Scattering (DLS). In addition, said inconsistencies with respect to the specific size and conformation of the formed aggregated oligonucleotides led further to substantial inconsistencies in the virus-like particles packaged with said aggregated oligonucleotides causing not only the formation of the desired spherical packaged VLPs, but in addition the formation of malformed rod-like aggregates or higher order aggregates. Moreover, said resulting VLPs packaged with said highly polydispersed aggregated oligonucleotides showed not only a lower purity but further an increased instability.

Importantly, it has further been found that the prior art disaggregation/aggregation process of WO2007/144150 is highly dependent on the initial purity of the oligonucleotides used for said disaggregation/aggregation steps. In particular, it has been found that the initial purity of the oligonucleotides used in the disaggregation/aggregation process of WO2007/144150 has an impact on the aggregation of the oligonucleotides, i.e. on the rate of aggregation and, thus, the G-quadruplex formation, to form the defined desired size and conformation of the aggregated oligonucleotides. Typically, the higher the purity of the oligonucleotides used in the disaggregation/aggregation process of WO2007/144150, the faster and more uncontrolled and chaotic the aggregation occurred resulting in an increased amount of typically very large aggregated oligonucleotides outside the desired size window. As a consequence, and since the resulting aggregated oligonucleotides cannot be properly packaged, the final oligonucleotides-packaged-VLPs had a decreased purity requiring intense and costly purification and a decreased stability as evidenced by the change in the SEC chromatogram overtime. Specifically, an increase in low molecular weight peaks were observed overtime suggesting that some of the VLPs were not stable and released the originally packaged DNA and oligonucleotides, respectively,

Based on the findings by the inventors and, in particular, based on the large dependency on the purity of the initially used oligonucleotides for the disaggregation/aggregation process of WO2007/144150, it appeared that the processes of WO2007/144150, in particular the disaggregation/aggregation processes of WO2007/144150, have been developed and optimized for lower purity G-rich oligonucleotides. As indicated, not only are high purity G-rich oligonucleotides nowadays ubiquitously available, but, furthermore, their use for pharmaceutical drugs is a prerequisite for regulatory approval. Moreover, a further substantial disadvantage of the prior art processes of WO2007/144150, beside its inconsistencies and large variations with respect to the specific size and conformation of the formed aggregated oligonucleotides depending on the purity of the initially used oligonucleotides, is the herewith associated very narrow time window to achieve the preparation of the aggregated oligonucleotides in the defined size range due to the uncontrolled and chaotic aggregation.

As a consequence and due to these occurring inconsistencies and variations and the strong dependency on the purity of the initially used oligonucleotides, the processes of WO2007/144150 are not suitable for manufacturing at scale, and in particular, not suitable for GMP manufacturing especially for clinical trial material where batch to batch consistency is critical.

Therefore, the present invention provides processes for producing a nucleotide composition comprising aggregated oligonucleotides and for producing a composition comprising a virus-like particle of an RNA bacteriophage and aggregated oligonucleotides packaged in said virus-like particle, thus avoiding or reducing the disadvantages of the prior art processes.

Thus, in a first aspect, the present invention provides for a process for producing a nucleotide composition comprising aggregated oligonucleotides said process comprising the steps of:
(a) providing oligonucleotides, wherein said oligonucleotides comprise at least one poly G stretch;
(b) denaturing said oligonucleotides, wherein said denaturing comprises the step of
   (i) incubating an aqueous solution I comprising said oligonucleotides and a chaotropic agent at a temperature I until the average diameter of said oligonucleotides is 1 nm or less, wherein said average diameter is determined by Dynamic Light Scattering (DLS), and wherein said temperature I is 75° C. to 99° C., and wherein preferably said chaotropic agent is urea;
(c) aggregating said oligonucleotides, wherein said aggregating comprises the steps of
   (i) incubating an aqueous solution II comprising said oligonucleotides having said average diameter of 1 nm or less obtained in step (b), a chaotropic agent and a cation at a temperature II to form said aggregated oligonucleotides, wherein said incubating is performed until the average diameter of said formed aggregated oligonucleotides is 6-16 nm, wherein said average diameter is determined by Dynamic Light Scattering (DLS), and wherein said temperature II is 75° C. to 99° C., and wherein preferably said chaotropic agent is urea;
   (ii) adjusting the temperature of said solution II to a temperature III, wherein said temperature III is below 40° C., preferably below 30° C.;
wherein said steps are preferably performed in the given order.

Advantageously, the inventive processes allow the control of the size of the formed aggregated oligonucleotides, and, hereby, the conformation of aggregated oligonucleotides, and as a consequence thereof, the consistent formation of highly pure, stable and well-formed, namely typically exclusively spherical VLPs packaged with oligonucleotides.

Thus, the inventive processes allow control of the size of the aggregated oligonucleotides by their diameter of between 6-16 nm, preferably 7-14 nm, further preferably 8-14 nm, again further preferably 9-14 nm, again further preferably 10-14 nm, again further preferably 11-13 nm, thus 11, 12 or 13 nm, and most preferably 12 nm, wherein said diameter is determined by Dynamic Light Scattering (DLS).

Importantly, the inventive processes allow not only control of the size and thus, the conformation of the formed aggregated oligonucleotides, but in addition to do so regardless of the purity of the oligonucleotides used for the denaturing step. Furthermore, the inventive processes further enable and provides a wider operating window in which to perform the aggregation step as it allows control of the aggregation. Such control and additional time and wider operating window, respectively, as well as the higher preciseness in the process controls, make the inventive processes highly beneficial for the costly production in GMP quality, in particular large-scale GMP quality. Moreover, the yield of the final obtained oligonucleotide-packaged-VLPs is additionally much higher and more pure, in particular, without the need of further costly purification steps.

As indicated, the prior art processes of WO2007/144150 led to the formation of aggregated oligonucleotides that showed substantial inconsistencies and large variations with respect to the specific size and conformation of the formed aggregated oligonucleotides, as determined by Dynamic Light Scattering (DLS), in contrast to the aggregated oligonucleotides formed by the present inventive processes. It has to be noted, however, that the aggregated oligonucleotides formed by the present inventive processes all met the specification criteria of the relative peak start time (PST), in size exclusion HPLC using the capsid of said RNA bacteriophage as standard, as defined in WO2007/144150, even though the corresponding optimal range is slightly shifted. Thus, the preferred aggregated oligonucleotides formed by the present inventive processes do possess a PST as determined accordingly of 90-105%, preferably of 92%-102%.

A further advantage of the inventive processes is the avoidance of salts and, in particular, the use of a chaotropic agent, preferably urea, for the denaturing step as compared to the prior art process of WO2007/144150. As a consequence, the resulting solutions of the inventive processes containing the denatured, typically monomeric, oligonucleotides are stable without the threat of re-aggregation and thus, could be stored for further use. As such, these solutions may be heated or cooled multiple times without forming aggregates, and advantageously, could be frozen for future use. The latter has not been possible for the prior art processes most likely as indicated due to the presence of salt produced during neutralization step required to stop the denaturing step prior to oligonucleotide degradation. Thus, said prepared prior art solutions had to be subsequently used without the possibility of storage.

For the present invention, the aggregation state of the oligonucleotide is characterized by Dynamic Light Scattering (DLS) which measures the time-dependent fluctuations in the scattered light. The hydrodynamic radii and diameters of the aggregated oligonucleotides are then calculated by relating the rate of diffusion of the aggregate through the solvent. Aggregated oligonucleotides comprising an average hydrodynamic diameter of 6-16 nm, preferably 7-14 nm, further preferably 8-14 nm, again further preferably 9-14 nm, again further preferably 10-14 nm, again further preferably 11-13 nm, thus, 11, 12 or 13 nm, and most preferably 12 nm, has been found to be optimal.

Thus, in a further aspect, the present invention provides for a process for producing a composition comprising (i) a virus-like particle, wherein said virus-like particle is a virus-like particle of an RNA bacteriophage, and (ii) aggregated oligonucleotides, wherein said aggregated oligonucleotides are packaged into said virus-like particle, said process comprising the steps of:
(a) generating a mixture, wherein said mixture comprises:
   (i) a coat protein of said RNA bacteriophage;
   (ii) an agent capable of preventing the self-assembly of said coat protein; and
   (iii) aggregated oligonucleotides, wherein said aggregated oligonucleotides comprise oligonucleotides comprising at least one poly G stretch, and wherein said aggregated oligonucleotides have an average diameter of 6-16 nm, wherein said average diameter is determined by Dynamic Light Scattering (DLS);
(b) removing said agent from said mixture; and
(c) allowing said coat protein to self-assemble into a virus-like particle and to package said aggregated oligonucleotides.

In an again further aspect, the present invention provides for a process for producing a composition comprising (i) a virus-like particle, wherein said virus-like particle is a virus-like particle of an RNA bacteriophage, and (ii) aggregated oligonucleotides, wherein said aggregated oligonucleotides are packaged into said virus-like particle, said process comprising the steps of:
(a) generating a mixture, wherein said mixture comprises:
   (i) a coat protein of said RNA bacteriophage;
   (ii) an agent capable of preventing the self-assembly of said coat protein; and
   (iii) aggregated oligonucleotides, wherein said aggregated oligonucleotides comprise oligonucleotides comprising at least one poly G stretch, and wherein said aggregated oligonucleotides are obtainable by the process according to the first aspect of the present invention, and wherein said aggregated oligonucleotides have an average diameter of 6-16 nm, wherein said average diameter is determined by Dynamic Light Scattering (DLS);
(b) removing said agent from said mixture; and
(c) allowing said coat protein to self-assemble into a virus-like particle and to package said aggregated oligonucleotides.

In an again further aspect, the present invention provides for a process for producing a composition comprising (i) a virus-like particle, wherein said virus-like particle is a virus-like particle of an RNA bacteriophage, and (ii) aggregated oligonucleotides, wherein said aggregated oligonucleotides are packaged into said virus-like particle, said process comprising the steps of:
(a) generating a mixture, wherein said mixture comprises:
   (i) a coat protein of said RNA bacteriophage;
   (ii) an agent capable of preventing the self-assembly of said coat protein; and
   (iii) the nucleotide composition comprising said aggregated oligonucleotides, and wherein said nucleotide composition is obtainable by the process for producing a nucleotide composition comprising aggregated oligonucleotides in accordance with the present invention, and wherein said aggregated oligonucleotides comprise oligonucleotides comprising at least one poly G stretch, and wherein said aggregated oligonucleotides have an average diameter of 6-16 nm, wherein said average diameter is determined by Dynamic Light Scattering (DLS);
(b) removing said agent from said mixture; and
(c) allowing said coat protein to self-assemble into a virus-like particle and to package said aggregated oligonucleotides.

During said processes said virus-like particle is formed by self-assembly of coat protein of said RNA bacteriophage in the presence of said aggregated oligonucleotides.

In an again further aspect, the present invention provides for a nucleotide composition comprising aggregated oligonucleotides, wherein said nucleotide composition is obtainable by the process for producing a nucleotide composition comprising aggregated oligonucleotides in accordance with the present invention, wherein preferably said aggregated oligonucleotides have an average diameter of 6-16 nm, preferably 7-14 nm, wherein said average diameter is determined by Dynamic Light Scattering (DLS).

In an again further aspect, the present invention provides for a nucleotide composition comprising aggregated oligonucleotides, wherein said aggregated oligonucleotides have an average diameter of 7-14 nm, wherein said average diameter is determined by Dynamic Light Scattering (DLS).

In an again further aspect, the present invention provides for a composition comprising (i) a virus-like particle of an RNA bacteriophage, and (ii) aggregated oligonucleotides, wherein said aggregated oligonucleotides are packaged into said virus-like particle, wherein said composition is obtainable by a process for producing a composition comprising (i) a virus-like particle, wherein said virus-like particle is a virus-like particle of an RNA bacteriophage, and (ii) aggregated oligonucleotides in accordance with the present invention.

In an again further aspect, the present invention provides for a composition comprising (i) a virus-like particle of an RNA bacteriophage, and (ii) aggregated oligonucleotides, wherein said aggregated oligonucleotides are packaged into said virus-like particle, wherein said aggregated oligonucleotides have an average diameter of 6-16, preferably of 7-14 nm, wherein said average diameter is determined by Dynamic Light Scattering (DLS).

Further aspects and embodiments of the present invention will be become apparent as this description continues.

### DESCRIPTION OF FIGURES

FIG. 1A: Dynamic Light Scattering (DLS) of denatured oligonucleotide G10 and aggregated oligonucleotides G10 as obtained by the inventive process. DLS was performed as described in Example 3. FIG. 1A shows denatured high purity G10 oligonucleotide with an average particle diameter of 0.90 nm indicating that the G10 oligonucleotide secondary structures have been disrupted, denaturing is complete, and monomers have been achieved. Multiple scans were performed as shown by the overlapping curves. The average diameter (D_{hyd}) and percent of primary peak (mean) of these scans are reported within data box inset in the graph.

FIG. 1B: Dynamic Light Scattering (DLS) of denatured oligonucleotide G10 and aggregated oligonucleotides G10 as obtained by the inventive process. DLS was performed as described in Example 3. FIG. 1B shows the subsequently aggregated G10 oligonucleotides obtained with proper aggregation and a diameter of 12 nm. Multiple scans were performed as shown by the overlapping curves. The average diameter (D_{hyd}) and percent of primary peak (mean) of these scans are reported within data box inset in the graph.

FIG. 2A: DLS of denatured oligonucleotides G10. DLS was performed as described in Example 3. Low purity oligonucleotide G10 of SEQ ID NO:1 (about 79% as determined by reverse phase HPLC and anion Exchange HPLC) was used for the disaggregation (denaturation) as described in the prior art (WO2007/144150). DLS shows particles with an average diameter of 2.2 nm indicating that not all oligonucleotide G10 secondary structure has been disrupted and denatured to monomer. Multiple scans were performed as shown by the overlapping curves. The average diameter (D_{hyd}) and percent of primary peak (mean) of these scans are reported within data box inset in the graph. FIG. 2B: DLS of denatured oligonucleotides G10. DLS was performed as described in Example 3. High purity oligonucleotide G10 of SEQ ID NO:1 (about 94% as determined by reverse phase HPLC and anion Exchange HPLC) was used for the disaggregation (denaturation) as described in the prior art (WO2007/144150). DLS shows particles with an average diameter of 2.8 nm indicating that not all oligonucleotide G10 secondary structure has been disrupted and denatured to monomer. Multiple scans were performed as shown by the overlapping curves. The average diameter (D_{hyd}) and percent of primary peak (mean) of these scans are reported within data box inset in the graph.

FIG. 2C: DLS of denatured oligonucleotides G10. DLS was performed as described in Example 3. High purity oligonucleotide G10 of SEQ ID NO:1 (about 94% as determined by reverse phase HPLC and anion Exchange HPLC) was used for the denaturation of the inventive process. DLS shows particles with an average diameter of 0.9 nm indicating that oligonucleotide G10 has been completely or substantially completely denatured to monomer. Multiple scans were performed as shown by the overlapping curves. The average diameter (D_{hyd}) and percent of primary peak (mean) of these scans are reported within data box inset in the graph.

FIG. 3A: DLS of aggregated oligonucleotides G10. DLS was performed as described in Example 3. Denatured oligonucleotides as obtained from Example 5 (FIG. 2A-2C) were used. Aggregation as described in the prior art (WO2007/144150) of low purity G10 denatured by prior art process. Low purity oligonucleotide G10 of SEQ ID NO:1 corresponds to about 79% purity as determined by reverse phase HPLC and anion Exchange HPLC. DLS shows aggregated oligonucleotides which are not only on the high side of the desired particle range (15 nm), but additionally, 10% of the material is significantly larger (30-50 nm). Multiple scans were performed as shown by the overlapping curves. The average diameter (D_{hyd}) and percent of primary peak (mean) of these scans are reported within data box inset in the graph.

FIG. 3B: DLS of aggregated oligonucleotides G10. DLS was performed as described in Example 3. Denatured oligonucleotides as obtained from Example 5 (FIG. 2A-2C) were used. Aggregation as described in the prior art (WO2007/144150) of high purity G10 denatured by prior art process. High purity oligonucleotide G10 of SEQ ID NO: XX corresponds to about 94% purity as determined by reverse phase HPLC and anion Exchange HPLC. DLS shows aggregated oligonucleotides with an average diameter being completely (100%) outside the diameter range of 6-16 nm (the desired range) as illustrated by the shaded box. Multiple scans were performed as shown by the overlapping curves. The average diameter (D_{hyd}) and percent of primary peak (mean) of these scans are reported within data box inset in the graph.

FIG. 3C: DLS of aggregated oligonucleotides G10. DLS was performed as described in Example 3. Denatured oligonucleotides as obtained from Example 5 (FIG. 2A-2C) were used. Aggregation of high purity G10 in accordance with the present inventive process. High purity oligonucleotide G10 of SEQ ID NO:1 corresponds to about 94% purity as determined by reverse phase HPLC and anion Exchange HPLC. DLS shows aggregated oligonucleotides with an average diameter of said aggregated oligonucleotides being completely (100%) within the diameter range of 6-16 nm (the desired range) as illustrated by the shaded box. Multiple scans were performed as shown by the overlapping curves. The average diameter (D_{hyd}) and percent of primary peak (mean) of these scans are reported within data box inset in the graph.

FIG. 4A: Characterization of purified Qβ coat protein by analytical size exclusion chromatography. Sample of purified Qβ VLP. The observed peak (ratio A260/A280=2) is dominated by the RNA core of the VLP, because the absorption coefficient of RNA at 260 nm is approx. 100 fold higher than the absorption coefficient of the coat protein.

FIG. 4B: Characterization of purified Qβ coat protein by analytical size exclusion chromatography. Sample of the supernatant of the disassembly reaction. Released coat protein is indicated by the presence of the protein-like peak at approx. 12 min. Furthermore several species of non-precipitated RNA molecules are present in the range 6.8 to 11 min.

FIG. 4C: Characterization of purified Qβ coat protein by analytical size exclusion chromatography. Sample of purified Qβ coat protein. Analysis was performed in PBS on column TSK G5000PWx1 (Tosoh Bioscience).

FIG. 5A: DLS and Electron Micrographs (EM) pictures of virus-like particle (VLP) of RNA bacteriophage Qβ packaged with aggregated oligonucleotides G10 obtained by the disaggregation-aggregation method of prior art and by the denaturation and aggregation of the present invention. DLS was performed as described in Example 3 and EM was taken as described in Example 8. DLS of QBVLPs packaged with aggregated oligonucleotides G10 obtained by the disaggregation-aggregation method of the prior art. Multiple scans were performed as shown by the overlapping curves. The average diameter (D_{hyd}) and percent of primary peak (mean) of these scans are reported within data box inset in the graph.

FIG. 5B: DLS and Electron Micrographs (EM) pictures of virus-like particle (VLP) of RNA bacteriophage Qβ packaged with aggregated oligonucleotides G10 obtained by the disaggregation-aggregation method of prior art and by the denaturation and aggregation of the present invention. DLS was performed as described in Example 3 and EM was taken as described in Example 8. EM of QBVLPs packaged with aggregated oligonucleotides G10 obtained by the disaggregation-aggregation method of the prior art. Arrows included to identify the rod like structures.

FIG. 5C: DLS and Electron Micrographs (EM) pictures of virus-like particle (VLP) of RNA bacteriophage Qβ packaged with aggregated oligonucleotides G10 obtained by the disaggregation-aggregation method of prior art and by the denaturation and aggregation of the present invention. DLS was performed as described in Example 3 and EM was taken as described in Example 8. DLS of QBVLPs packaged with aggregated oligonucleotides G10 obtained by the inventive process. Multiple scans were performed as shown by the overlapping curves. The average diameter (D_{hyd}) and percent of primary peak (mean) of these scans are reported within data box inset in the graph.

FIG. 5D: DLS and Electron Micrographs (EM) pictures of virus-like particle (VLP) of RNA bacteriophage Qβ packaged with aggregated oligonucleotides G10 obtained by the disaggregation-aggregation method of prior art and by the denaturation and aggregation of the present invention. DLS was performed as described in Example 3 and EM was taken as described in Example 8. EM of QBVLPs packaged with aggregated oligonucleotides G10 obtained by the inventive process.

### DETAILED DESCRIPTION OF THE INVENTION

Unless defined otherwise, all technical and scientific terms used herein have the same meanings as commonly understood by one of ordinary skill in the art to which this invention belongs.

Average diameter: The term “average diameter” as determined by Dynamic Light Scattering (DLS), as used herein, refers to a diameter as measured typically and preferably by DLS in the manner as described in Example 3, and which given average diameter value of measured particles, refers to particles having said diameter as the mean in a normal distribution (Gaussian distribution). Thus, the term “average diameter” as determined by Dynamic Light Scattering (DLS), as used herein, and as typically and preferably applied to the oligonucleotides, aggregated oligonucleotides and VLPs packaged with aggregated oligonucleotides in accordance with the present invention, typically and preferably refers to a diameter as measured by DLS, typically and preferably in the manner as described in Example 3, and which given average diameter value of measured particles, refers to particles, wherein at least 90%, preferably at least 95%, of said particles have a diameter of said given value or a diameter of ±10% of said given value. By way of clarification, for example, an average diameter value of 12 nm of the inventive aggregated oligonucleotides refers to said inventive aggregated oligonucleotides, wherein at least 90%, preferably at least 95%, of said inventive aggregated oligonucleotides have a diameter of 10.8 nm to 13.2 nm. Further, the term “average diameter” as determined by Dynamic Light Scattering (DLS), as used herein, and as typically and preferably applied to the oligonucleotides, aggregated oligonucleotides and VLPs packaged with aggregated oligonucleotides in accordance with the present invention, typically and preferably refers to a diameter as measured by DLS, typically and preferably in the manner as described in Example 3, and which given average diameter value of measured particles, refers to particles, wherein at least 65%, preferably at least 70%, of said particles have a diameter of said given value or a diameter of ±5% of said given value. By way of clarification, for example, an average diameter value of 12 nm of the inventive aggregated oligonucleotides refers to said inventive aggregated oligonucleotides, wherein at least 65%, preferably at least 70%, of said inventive aggregated oligonucleotides have a diameter of 11.4 nm to 12.6 nm.

All ranges of values, in particular all ranges of average diameters or diameters disclosed herein, should refer to all values falling within said range including the values defining the range. By way of clarification, for example, a diameter value of 12 nm to 13 nm should refer to a diameter of 12nm or 13 nm or all diameters falling within 12 nm and 13 nm.

Chaotropic agent: The term “chaotropic agent” as used herein refers to a molecule or substance that disrupts the ordered structure of a protein, oligonucleotide, or other macromolecule. This decrease in stability is typically caused by the disruption of the hydrogen bonding network. Examples include urea, phenol, isopropyl alcohol (IPA), ethanol and guanidinium chloride amongst others.

Oligonucleotide: The term oligonucleotide as used herein refers to a single stranded deoxyribonucleotide. A preferred oligonucleotide comprises at least one poly G stretch as defined below. More preferred oligonucleotides comprise 2, 3, 4, 5 or 6 of said poly G stretches. Very preferred oligonucleotides comprise exactly two poly G stretches, wherein preferably one of said two poly G stretches is located at the 5′ end or at the 3′ end of said oligonucleotide. Even more preferred oligonucleotides comprise exactly two poly G stretches, wherein one of said two poly G stretches is located at the 5′ end of said oligonucleotide and one of said two poly G stretches is located at the 3′ end of said oligonucleotide. Typically and preferably, an oligonucleotide as used herein consists of 6 to 1000, preferably of 10 to 1000, more preferably of 10 to 200, still more preferably of 10 to 100, still more preferably of 20 to 40, and most preferably of 30 nucleotides. Further preferred oligonucleotides consist of 15, 16, 17, 18, 19, 20, 21, 22, 23, 24, 25, 26, 27, 28, 29, 30, 31, 32, 33, 34, 35, 36, 37, 38, 39, or 40 nucleotides. Still more preferred oligonucleotides consists of 24 to 32 nucleotides, more preferably of about 30 nucleotides.

The term oligonucleotide also refers to molecules comprising at least one modified nucleotide, wherein preferably said modified nucleotide is selected from (a) a nucleotide analogue or (b) a nucleotide comprising a backbone modification. In one embodiment the oligonucleotide comprises at least one modified nucleotide selected from the group consisting of (a) peptide nucleic acid, (b) inosin, (c) tritylated bases, (d) phosphorothioates, (c) alkylphosphorothioates, (f) 5-nitroindole desoxyribofuranosyl, (g) 5-methyldesoxycytosine, and (h) 5,6-dihydro-5,6-dihydroxydesoxythymidine. In a further embodiment the oligonucleotide comprises or alternatively consists of phosphorothioated nucleotides. Phosphorothioated nucleotides are protected against degradation in a cell or an organism and are therefore preferred nucleotide modifications. Further preferred are chemically, enzymatically or metabolically modified forms of polynucleotides as typically found in nature. However, preferred oligonucleotides exclusively consists of unmodified nucleotides, i.e. of adenosine, tymidine, guanosine, and/or cytidine. Still further preferred oligonucleotides exclusively consists of phosphodiester bound nucleotides.

Very preferred oligonucleotides are unmethylated CpG containing oligonucleotides comprising at least one, preferably one, two, three or four CpG motifs. Still more preferred oligonucleotides comprise a palindromic sequence, wherein preferably said palindromic sequence comprises least one, preferably one, two, three or four CpG motifs. Still more preferred oligonucleotides comprise a palindromic sequence, wherein preferably said palindromic sequence comprises, or preferably consists of the sequence GACGATCGTC (SEQ ID NO:2). Still more preferred oligonucleotides comprise a palindromic sequence, wherein said palindromic sequence is flanked at its 5′ end by a poly G stretch and wherein said palindromic sequence is flanked at its 3′ end by a poly G stretch, wherein preferably said palindromic sequence is GACGATCGTC (SEQ ID NO:2). Very preferred oligonucleotides comprise a palindromic sequence, wherein said palindromic sequence is flanked at its 5′ end by at least 3 to 15, preferably by 6 to 10 guanosine entities and wherein said palindromic sequence is flanked at its 3′ end at least 3 to 15, preferably by 6 to 10, guanosine entities, wherein preferably said palindromic sequence is GACGATCGTC (SEQ ID NO:2).

Poly G stretch: The term poly G stretch relates to a segment of an oligonucleotide, wherein said segment consists of at least 3 consecutive guanosine residues. Preferred poly G stretches consist of 3 to 25, preferably of 4 to 20, more preferably of 4 to 15 and most preferably of 4 to 10 consecutive guanosine entities. Further preferred poly G stretches consist of 3, 4, 5, 6, 7, 8, 9, 10, 11, 12, 13, 14, 15, 16, 17, 18, 19, or 20 consecutive guanosine entities.

CpG motif: As used herein, the term CpG motif refers to short DNA sequence, preferably a single stranded DNA sequence, comprising a cytosine (C)—guanosine (G) dinucleotide, wherein C is unmethylated and wherein preferably said CG dinucleotide is phosphodiester bound. Preferably, a CpG motif comprises at least one, preferably one, two or three, additional nucleotides 5′ and/or 3′ of said CG dinucleotide, wherein further preferably said additional nucleotides do not comprise a CG dinucleotide.

Relative peak start time: The term “relative peak start time” is a parameter which is indicative of the aggregation state of an oligonucleotide and which was analyzed essentially as described in WO 2007/144150 by analytical size exclusion HPLC using the conditions as described in Example 4.

Packaged: The term “packaged” as used herein refers to the state of an oligonucleotide, typically and preferably of aggregated oligonucleotides, in relation to the virus-like particle. The use of the terms “aggregated oligonucleotides packaged into VLP” or “VLP packaged with aggregated oligonucleotides” is equivalent. The term “packaged” as used herein typically and preferably refers to non-covalent binding, preferably to ionic interactions, hydrophobic interactions, or hydrogen bonds. Typically and very preferably, the term “packaged” as used herein refers to the encapsulation of said aggregated oligonucleotides within the VLP. Typically and preferably, a VLP packaged with aggregated oligonucleotides protects said aggregated oligonucleotides from degradation, preferably from DNAse hydrolysis. Therefore, in the preferred meaning, the term “packaged” indicates that the aggregated oligonucleotides in a packaged state are not accessible to DNAse hydrolysis. More preferably, the term “packaged” indicates that the aggregated oligonucleotides are not accessible to DNAse hydrolysis, wherein further preferably the DNAse is DNAseI or Benzonase. Still more preferably, the term “packaged” indicates that the aggregated oligonucleotides are not accessible to Benzonase hydrolysis.

The accessibility of the oligonucleotide for DNAse (e.g. DNaseI or Benzonase) is preferably assayed as described in Examples 11-17 of WO2003/024481A2 (see p. 111 therein). In a preferred meaning, a VLP is regarded as being packaged with an oligonucleotide, when after treatment with Benzonase (190 U Benzonase/mg coat protein in a buffer comprising 2 mM MgCl₂, pH 7.2, 20-25° C., 18 h) at least 90%, preferably at least 95%, most preferably at least 98% of said oligonucleotide can be recovered from said VLP (e.g. in an ethidiumbromide stained gel). It is apparent for the artisan that such assays require appropriate controls and may need to be adapted to the specific combination of VLP and oligonucleotide. In a very preferred meaning, oligonucleotide G10 (SEQ ID NO:1) is regarded as being packaged into a VLP of an RNA bacteriophage Qβ, when after treatment with Benzonase (190 U Benzonase/mg coat protein in a buffer comprising 2 mM MgCl₂, pH 7.2, 20-25° C., 18 h) at least 90%, preferably at least 95%, most preferably at least 98% of said G10 can be recovered from said VLP of RNA bacteriophage Qβ.

Coat protein: As used herein, the term “coat protein” refers to the protein(s) of a RNA bacteriophage capable of being incorporated within the capsid assembly of the bacteriophage or the RNA bacteriophage. Thus, the term coat protein refers to the protein forming the capsid of a RNA bacteriophage or a VLP of a RNA bacteriophage. Typically and preferably, coat protein of RNA bacteriophages has a dimeric structure.

Fragment of a recombinant coat protein: Fragment of a recombinant coat protein, as used herein, is defined as a polypeptide, which is of at least 70%, preferably at least 80%, more preferably at least 90%, even more preferably at least 95% the length of the wild-type coat protein, or wild type recombinant protein, respectively and which preferably retains the capability of forming VLP. Preferably the fragment is obtained by at least one internal deletion, at least one truncation or at least one combination thereof. The term “fragment of a recombinant coat protein” or “fragment of a coat protein” shall further encompass polypeptide, which has at least 80%, preferably 90%, even more preferably 95% amino acid sequence identity with the wildtype coat protein, respectively, and which is preferably capable of assembling into a virus-like particle. The term “mutant coat protein” refers to a polypeptide having an amino acid sequence derived from the wild type recombinant protein, or coat protein, respectively, wherein the amino acid sequence is at least 80%, preferably at least 85%, 90%, 95%, 97%, or 99% identical to the wild type sequence and preferably retains the ability to assemble into a VLP.

Virus-like particle (VLP): VLP as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. Preferably a virus-like particle in accordance with the invention is non-replicative and/or non-infectious since it lacks all or part of the viral genome or genome function. In one embodiment, a virus-like particle is a virus particle, in which the viral genome has been physically or chemically inactivated, removed by disassembly and reassembly, or by assembly of purified proteins into a VLP. Typically and more preferably a virus-like particle lacks all or part of the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, preferably RNA bacteriophage. The term “capsid”, refers to a macromolecular assembly composed of viral protein subunits. Typically, there are 60, 120, 180, 240, 300, 360 and more than 360 viral protein subunits. Typically and preferably, the interactions of these subunits lead to the formation of viral capsid with an inherent repetitive organization, wherein said structure typically and preferably is spherical. For example, the capsids of RNA bacteriophages have a spherical form of icosahedral symmetry.

Virus-like particle of an RNA bacteriophage: As used herein, the term “virus-like particle of a RNA bacteriophage” refers to a virus-like particle comprising, or preferably consisting essentially of or consisting of coat proteins, mutants or fragments thereof, of a RNA bacteriophage. In addition, virus-like particle of a RNA bacteriophage resembling the structure of a RNA bacteriophage, being non replicative and/or non-infectious, and lacking at least the gene or genes encoding for the replication machinery of the RNA bacteriophage, and typically also lacking the gene or genes encoding the protein or proteins responsible for viral attachment to or entry into the host. Preferred VLPs derived from RNA bacteriophages exhibit icosahedral symmetry and consist of 180 subunits. In the context of the invention the term virus-like particle of an RNA bacteriophage preferably relates to a macromolecular structure obtained by the self-assembly of recombinant coat protein of an RNA bacteriophage, or fragments or mutants thereof, wherein preferably said self-assembly took place in the presence of oligonucleotide and aggregated oligonucleotides, respectively.

Agent capable of preventing the self assembly of coat protein: An agent capable of preventing the self assembly of coat protein is an agent which prevents the spontaneous formation of virus-like particles in said mixture. The artisan is able to determine the chemical nature and the appropriate concentration of said agent experimentally, e.g. by analyzing said mixture by size exclusion chromatography, as for example disclosed in Example 9 of WO2007/144150. An agent is capable of preventing the self assembly of coat protein, when after incubation of said mixture for at least 1 h at room temperature, preferably at 22° C., no virus-like particle is detectable by the size exclusion chromatography, as for example disclosed in Example 9 of WO2007/144150. However, agent which is capable of preventing the self assembly of coat protein, does not irreversibly modify said coat protein and removing said agent from said mixture will result in the spontaneous formation of virus-like particles. Preferred agents capable of preventing the self assembly of coat protein comprise detergents, guanidinium hydrochloride and urea, most preferably urea. Preferred detergents are sodium dodecyl sulfate, Tween 20, TritonX 100 and the like. Typically and preferably agents capable of preventing the self assembly of coat protein further comprise a reducing agent such as typically and preferably DDT which keeps intermolecular disulfide bounds formed by cysteine residues of said coat protein in a reduced state.

Purity: The purity of a composition of the invention comprising (i) a virus-like particle, wherein said virus-like particle is a virus-like particle of an RNA bacteriophage, and (ii) aggregated oligonucleotides, wherein said aggregated oligonucleotides are packaged into said virus-like particle, is determined by analytic size exclusion HPLC, wherein said HPLC is performed under conditions essentially, preferably exactly as disclosed in Example 4. The purity of said composition is determined as the percentage of the peak area of said virus-like particle contained in said composition relative to the total peak area of all peaks of the same chromatogram.

One”, “a/an”: When the terms “one,” “a,” or “an” are used in this disclosure, they mean “at least one” or “one or more,” unless otherwise indicated.

About: within the meaning of the present application the expression about shall have the meaning of +/−4%, typically and preferably of +/−2%. For example about 100 shall mean 96 to 104, typically and preferably 98 to 102.

The present invention provides for a process for producing a nucleotide composition comprising aggregated oligonucleotides said process comprising the steps of: (a) providing oligonucleotides, wherein said oligonucleotides comprise at least one poly G stretch; (b) denaturing said oligonucleotides, wherein said denaturing comprises the step of (i) incubating an aqueous solution I comprising said oligonucleotides and a chaotropic agent at a temperature I until the average diameter of said oligonucleotides is 1 nm or less, wherein preferably said average diameter is determined by Dynamic Light Scattering (DLS), and wherein said temperature I is 75° C. to 99° C., and wherein preferably said chaotropic agent is urea; (c) aggregating said oligonucleotides, wherein said aggregating comprises the steps of (i) incubating an aqueous solution II comprising said oligonucleotides having said average diameter of 1 nm or less obtained in step (b), a chaotropic agent and a cation at a temperature II to form said aggregated oligonucleotides, wherein said incubating is performed until the average diameter of said formed aggregated oligonucleotides is 6-16 nm, wherein preferably said average diameter is determined by Dynamic Light Scattering (DLS), and wherein said temperature II is 75° C. to 99° C., and wherein preferably said chaotropic agent is urea; (ii) adjusting the temperature of said solution II to a temperature III, wherein said temperature III is below 40° C., preferably below 30° C.; wherein said steps are preferably performed in the given order.

The present invention further provides a process for producing aggregated oligonucleotides, wherein said process comprises the steps of: (a) providing oligonucleotides, wherein said oligonucleotides comprise at least one poly G stretch; (b) denaturing said oligonucleotides, wherein said denaturing comprises the step of (i) incubating an aqueous solution I comprising said oligonucleotides and a chaotropic agent at a temperature I until the average diameter of said oligonucleotides is 1 nm or less, wherein preferably said average diameter is determined by Dynamic Light Scattering (DLS), and wherein said temperature I is 75° C. to 99° C., and wherein preferably said chaotropic agent is urea; (c) aggregating said oligonucleotides, wherein said aggregating comprises the steps of (i) incubating an aqueous solution II comprising said oligonucleotides having said average diameter of 1 nm or less obtained in step (b), a chaotropic agent and a cation at a temperature II to form said aggregated oligonucleotides, wherein said incubating is performed until the average diameter of said formed aggregated oligonucleotides is 6-16 nm, wherein preferably said average diameter is determined by Dynamic Light Scattering (DLS), and wherein said temperature II is 75° C. to 99° C., and wherein preferably said chaotropic agent is urea; (ii) adjusting the temperature of said solution II to a temperature III, wherein said temperature III is below 40° C., preferably below 30° C.; wherein said steps are preferably performed in the given order.

In a preferred embodiment, said denaturing said oligonucleotides comprises the step of solubilizing said oligonucleotides in an aqueous solution comprising said chaotropic agent to form said aqueous solution I, wherein said aqueous solution does not comprise mono or divalent ions in a concentration higher than 1 mM, and wherein preferably said aqueous solution does not comprise mono or divalent ions in a concentration higher than 500 μM, preferably not higher than 250 μM, preferably not higher than 100 μM, preferably not higher than 50 μM, preferably not higher than 10 μM.

In a further preferred embodiment, said denaturing said oligonucleotides comprises the step of solubilizing said oligonucleotides in an aqueous solution comprising said chaotropic agent to form said aqueous solution I, wherein said aqueous solution does not comprise mono or divalent ions in a concentration which after the addition of the oligonucleotide would cause said oligonucleotides self-aggregate.

In a further preferred embodiment, said aqueous solution I does not comprise mono or divalent ions in a concentration such that said oligonucleotides self-aggregate.

In a further preferred embodiment, said denaturing said oligonucleotides comprises the step of solubilizing said oligonucleotides in an aqueous solution comprising said chaotropic agent to form said aqueous solution I, wherein said aqueous solution does not comprise mono or divalent ions in a concentration which after the addition of the oligonucleotide would cause said oligonucleotides spontaneously self-aggregate.

In a further preferred embodiment, said aqueous solution I does not comprise mono or divalent ions in a concentration such that said oligonucleotides spontaneously self-aggregate.

In a further preferred embodiment, said denaturing said oligonucleotides comprises the step of solubilizing said oligonucleotides and said chaotropic agent to form said aqueous solution I, and adjusting the temperature of said solution I to temperature I.

In a further preferred embodiment, said chaotropic agent comprised in said solution I is selected from urea, phenol, isopropyl alcohol, ethanol and guanidinium chloride.

In a further preferred embodiment, said chaotropic agent comprised in said solution I is urea.

In a further preferred embodiment, said temperature I is 75° C. to 90° C., preferably 80° C. to 90° C., further preferably 83° C. to 87° C., again further preferably about 85° C., and most preferably 85° C.

In a further preferred embodiment, said incubating said oligonucleotide in said solution I at said temperature I is performed for 10 to 120 min, preferably for 20 to 60 min, further preferably for 20 to 30 min, and again further preferably for 15-18 min.

In a further preferred embodiment, said concentration of said chaotropic agent, preferably said urea, in said solution I is 200nM to 5M, preferably 500 mM to 2M, further preferably 500 mM to 1.5M, and again further preferably 1M.

In a further preferred embodiment, said concentration of said oligonucleotides, preferably said oligonucleotides of SEQ ID NO:1, in said solution I is 100 μM to 1 mM, preferably 100 μM to 750 μM, further preferably 200 μM to 600 μM, and again further preferably 350 μM to 500 μM.

In a further preferred embodiment, said incubating said oligonucleotides in said solution I at said temperature I is performed between 15 minutes and 120 minutes, preferably between 15 min to 60 min, and further preferably between 15 min to 30 min, again further preferably between 15 min to 25 min.

In a further preferred embodiment, said oligonucleotides comprise at its 5′ end at least 3 and at most 15 guanosine entities and at its 3′ end at least 3 and at most 15 guanosine entities, preferably at least 6 and at most 13 guanosine entities and at its 3′ end at least 6 and at most 13 guanosine entities, further preferably at least 8 and at most 11 guanosine entities and at its 3′ end at least 8 and at most 11 guanosine entities.

In a further preferred embodiment, said oligonucleotides comprise a palindromic sequence, wherein preferably said palindromic sequence is GACGATCGTC (SEQ ID NO:2), and wherein further preferably said palindromic sequence is flanked at its 5′ end by at least 3 and at most 15 guanosine entities and wherein said palindromic sequence is flanked at its 3′ end by at least 3 and at most 15 guanosine entities, and wherein again further preferably said palindromic sequence is flanked at its 5′ end by at least 6 and at most 13 guanosine entities and wherein said palindromic sequence is flanked at its 3′ end by at least 6 and at most 13 guanosine entities, and wherein again further preferably said palindromic sequence is flanked at its 5′ end by at least 8 and at most 11 guanosine entities and wherein said palindromic sequence is flanked at its 3′ end by at least 8 and at most 11 guanosine entities.

In a further preferred embodiment, said oligonucleotides comprises 10 to 1000 nucleotides, preferably 10 to 200 nucleotides, further preferably 10 to 100 nucleotides, still further preferably 20 to 40 nucleotides, and still further preferably 30 nucleotides.

In a further preferred embodiment, said oligonucleotides comprise the nucleic acid sequence selected from the group consisting of:

(a) G10:
(SEQ ID NO: 1)
GGGGGGGGGGGACGATCGTCGGGGGGGGGG;
(b) G10-11:
(SEQ ID NO: 3)
GGGGGGGGGGGACGATCGTCGGGGGGGGGGG;
(c) G12-11:
(SEQ ID NO: 4)
GGGGGGGGGGGGGACGATCGTCGGGGGGGGGGG
(d) G6:
(SEQ ID NO: 5)
GGGGGGGACGATCGTCGGGGGG;
(e) G7:
(SEQ ID NO: 6)
GGGGGGGGACGATCGTCGGGGGGG;
(f) G8:
(SEQ ID NO: 7)
GGGGGGGGGACGATCGTCGGGGGGGG;
(g) G9:
(SEQ ID NO: 8)
GGGGGGGGGGACGATCGTCGGGGGGGGG;
(h) G11:
(SEQ ID NO: 9)
GGGGGGGGGGGGACGATCGTCGGGGGGGGGGG
(i) G6-10:
(SEQ ID NO: 24)
GGGGGGGACGATCGTCGGGGGGGGGG;
(j) G7-10:
(SEQ ID NO: 25)
GGGGGGGGACGATCGTCGGGGGGGGGG;
(k) G8-10:
(SEQ ID NO: 26)
GGGGGGGGGACGATCGTCGGGGGGGGGG;
and
(l) G9-10:
(SEQ ID NO: 27)
GGGGGGGGGGACGATCGTCGGGGGGGGGG.

In a further preferred embodiment, said oligonucleotides have the nucleic acid sequence

G10
(SEQ ID NO: 1)
GGGGGGGGGGGACGATCGTCGGGGGGGGGG.

In a further preferred embodiment, said oligonucleotides exclusively consists of phosphodiester connected deoxynucleotides.

In a further preferred embodiment, the purity of said oligonucleotides, preferably the purity of said oligonucleotides of SEQ ID NO:1, is 90% or higher, as determined by HPLC, preferably by reverse phase HPLC or anion Exchange HPLC, more preferably by reverse phase HPLC.

In a further preferred embodiment, the purity of said oligonucleotides, preferably the purity of said oligonucleotides of SEQ ID NO:1, is 92% or higher, preferably 94% or higher, further preferably 95% or higher, again further preferably 97% or higher, again further preferably 98% or higher, again further preferably 99% or higher, as determined by HPLC, preferably by reverse phase HPLC or anion Exchange HPLC, more preferably by reverse phase HPLC.

In a further preferred embodiment, said oligonucleotides comprise the sequence of SEQ ID NO:1, wherein said oligonucleotides exclusively consists of phosphodiester connected deoxynucleotides.

In a further preferred embodiment, said oligonucleotides consists of the sequence of SEQ ID NO:1.

In a further preferred embodiment, said oligonucleotides consists of the sequence of SEQ ID NO:1, wherein said oligonucleotides exclusively consists of phosphodiester connected deoxynucleotides.

In a further preferred embodiment, said chaotropic agent comprised in said solution II is selected from urea, phenol, isopropyl alcohol, ethanol and guanidinium chloride.

In a further preferred embodiment, said chaotropic agent comprised in said solution II is urea.

Thus, in a further preferred embodiment, said chaotropic agent comprised in said solution II, i.e. in said solution for aggregation, is urea.

In a further preferred embodiment, said temperature II is 75° C. to 90° C., preferably 80° C. to 90° C., further preferably 83° C. to 87° C., again further preferably about 85° C., and most preferably 85° C.

In a further preferred embodiment, said concentration of said chaotropic agent, preferably said urea, in said solution II is 200nM to 5M, preferably 500 mM to 2M, further preferably 500 mM to 1.5M, and again further preferably 1M.

In a further preferred embodiment, said cation is selected from Na³⁰ , K³⁰ , NH₄³⁰ , Li³⁰ , Ca²⁺, Mg²⁺and Zn²⁺.

Said cation is typically and preferably provided by way of an inorganic salt, and wherein further preferably said inorganic salt are selected from chlorides and sulfates. Preferably, said Na³⁰ , K³⁰ , NH₄³⁰ , Li³⁰ , Ca²⁺, Mg²⁺, Zn²⁺ as said cation are provided by its chloride salt. Alternatively, said Na⁺, K⁺, NH₄⁺, Li⁺, Ca²⁺, Mg²⁺, Zn²⁺ as said cation are provided by its sulfate salt. Again further preferably, said Na⁺, K⁺, NH₄⁺, Li⁺, Ca²⁺, Mg²⁺ as said cation are provided by its chloride salt, wherein said Zn²⁺ as said cation is preferably provided as its sulfate salt.

In a further preferred embodiment, said the concentration of said cation in said solution II is 20 mM to 2M, preferably 50 mM to IM, further preferably 100 mM to 500 mM, and again further preferably 250 mM.

In a further preferred embodiment, said chaotropic agent comprised in said solution I and said chaotropic agent comprised in said solution II is the same.

In a further preferred embodiment, the concentration of said chaotropic agent comprised in said solution I and said chaotropic agent comprised in said solution II is the same.

In a further preferred embodiment, said chaotropic agent comprised in said solution I and said chaotropic agent comprised in said solution II is urea.

In a further preferred embodiment, said aggregating said oligonucleotides comprises the steps of (i) solubilizing said chaotropic agent and said cation to form an aqueous solution IIa, (ii) mixing said aqueous solution IIa and said aqueous solution I comprising said oligonucleotides having said average diameter of 1 nm or less obtained in step (b) to form said aqueous solution II, and (iii) adjusting the temperature of said solution II to temperature II.

In a further preferred embodiment, the difference of temperature of said temperature I and said temperature II is 5° C. or less, preferably 4° C. or less, further preferably 3° C. or less, again further preferably 2° C. or less, again further preferably 1° C. or less, and most preferably said temperature I and said temperature II are equal.

In a further preferred embodiment, said incubating is performed until the average diameter of said aggregated oligonucleotides is 7-14 nm, wherein said average diameter is determined by Dynamic Light Scattering (DLS).

In a further preferred embodiment, said incubating is performed until the average diameter of said aggregated oligonucleotides is 8-14 nm, wherein said average diameter is determined by Dynamic Light Scattering (DLS).

In a further preferred embodiment, said incubating is performed until the average diameter of said aggregated oligonucleotides is 9-14 nm, wherein said average diameter is determined by Dynamic Light Scattering (DLS).

In a further preferred embodiment, said incubating is performed until the average diameter of said aggregated oligonucleotides is 10-14 nm, wherein said average diameter is determined by Dynamic Light Scattering (DLS).

In a further preferred embodiment, said incubating is performed until the average diameter of said aggregated oligonucleotides is 11-13 nm, wherein said average diameter is determined by Dynamic Light Scattering (DLS).

In a further preferred embodiment, said incubating is performed until the average diameter of said aggregated oligonucleotides is 11, 12 or 13 nm, wherein average said diameter is determined by Dynamic Light Scattering (DLS).

In a further preferred embodiment, said incubating is performed until the average diameter of said aggregated oligonucleotides is 12 nm, wherein said average diameter is determined by Dynamic Light Scattering (DLS).

The determination of the average diameter by Dynamic Light Scattering (DLS) in accordance with the present invention, be it that of said oligonucleotides in the course of the denaturing, or be it that of said aggregated oligonucleotides in the course of the aggregation, is highly beneficial since said determinations can be easily effected while the process is running. This further ensures a very high precision and very high control of the desired size of the oligonucleotides and aggregated oligonucleotides, respectively.

In a further preferred embodiment, said incubating is performed until said aggregated oligonucleotides comprise a relative peak start time of 80 to 110%, where said relative peak start time is determined by size exclusion HPLC with the capsid of an RNA bacteriophage as the standard.

In a further preferred embodiment, said process further comprises the step of purifying said aggregated oligonucleotides, and wherein preferably said purifying comprises filtering said aggregated oligonucleotides, preferably said solution II comprising said aggregated oligonucleotides, through a 50 nm filter.

In a further preferred embodiment, said 50 nm filter is a 50 nm PTFE filter.

In a further preferred embodiment, said filtering said aggregated oligonucleotides, preferably said solution II comprising said aggregated oligonucleotides, through said 50 nm filter, preferably said 50 nm PTFE filter, is performed at 0° C.-20° C.

Said further preferred step of purifying, preferably filtering, allows to remove any large aggregates prior to the packaging step into the VLPs and, thus, further leads typically to an even further increase in purity of the final product, i.e. the inventive VLPs packaged with the aggregated oligonucleotides by typically around 5%. The increase in purity is, thus, typically associated with a decrease in higher molecular weight material in the final product as evidenced by SEC HLPC or DLS.

In a further preferred embodiment, said process does not comprise a step of purifying said aggregated oligonucleotides.

In a further preferred embodiment, said aggregated oligonucleotides comprise at its 5′ end at least 3 and at most 15 guanosine entities and at its 3′ end at least 3 and at most 15 guanosine entities, preferably at least 6 and at most 13 guanosine entities and at its 3′ end at least 6 and at most 13 guanosine entities, further preferably at least 8 and at most 11 guanosine entities and at its 3′ end at least 8 and at most 11 guanosine entities.

In a further preferred embodiment, said aggregated oligonucleotides comprise a palindromic sequence, wherein preferably said palindromic sequence is GACGATCGTC (SEQ ID NO:2), and wherein further preferably said palindromic sequence is flanked at its 5′ end by at least 3 and at most 15 guanosine entities and wherein said palindromic sequence is flanked at its 3′ end by at least 3 and at most 15 guanosine entities, and wherein again further preferably said palindromic sequence is flanked at its 5′ end by at least 6 and at most 13 guanosine entities and wherein said palindromic sequence is flanked at its 3′ end by at least 6 and at most 13 guanosine entities, and wherein again further preferably said palindromic sequence is flanked at its 5′ end by at least 8 and at most 11 guanosine entities and wherein said palindromic sequence is flanked at its 3′ end by at least 8 and at most 11 guanosine entities.

In a further preferred embodiment, said aggregated oligonucleotides comprises 10 to 1000 nucleotides, preferably 10 to 200 nucleotides, further preferably 10 to 100 nucleotides, still further preferably 20 to 40 nucleotides, and still further preferably 30 nucleotides.

In a further preferred embodiment, said aggregated oligonucleotides comprise a nucleic acid sequence selected from the group consisting of:

(a) G10:
(SEQ ID NO: 1)
GGGGGGGGGGGACGATCGTCGGGGGGGGGG;
(b) G10-11:
(SEQ ID NO: 3)
GGGGGGGGGGGACGATCGTCGGGGGGGGGGG;
(c) G12-11:
(SEQ ID NO: 4)
GGGGGGGGGGGGGACGATCGTCGGGGGGGGGGG
(d) G6:
(SEQ ID NO: 5)
GGGGGGGACGATCGTCGGGGGG;
(e) G7:
(SEQ ID NO: 6)
GGGGGGGGACGATCGTCGGGGGGG;
(f) G8:
(SEQ ID NO: 7)
GGGGGGGGGACGATCGTCGGGGGGGG;
(g) G9:
(SEQ ID NO: 8)
GGGGGGGGGGACGATCGTCGGGGGGGGG;
(h) G11:
(SEQ ID NO: 9)
GGGGGGGGGGGGACGATCGTCGGGGGGGGGGG
(i) G6-10:
(SEQ ID NO: 24)
GGGGGGGACGATCGTCGGGGGGGGGG;
(j) G7-10:
(SEQ ID NO: 25)
GGGGGGGGACGATCGTCGGGGGGGGGG;
(k) G8-10:
(SEQ ID NO: 26)
GGGGGGGGGACGATCGTCGGGGGGGGGG;
and
(l) G9-10:
(SEQ ID NO: 27)
GGGGGGGGGGACGATCGTCGGGGGGGGGG.

In a further preferred embodiment, said aggregated oligonucleotides have the nucleic acid sequence GGGGGGGGGGGACGATCGTCGGGGGGGGGG (SEQ ID NO:1) (G10).

In a further preferred embodiment, said aggregated oligonucleotides exclusively consists of phosphodiester connected deoxynucleotides.

In a further aspect, the present invention provides for a nucleotide composition comprising aggregated oligonucleotides, wherein said nucleotide composition is obtainable by the process for producing a nucleotide composition comprising aggregated oligonucleotides in accordance with the present invention, wherein preferably said aggregated oligonucleotides have an average diameter of 6-16nm, preferably 7-14 nm, wherein said average diameter is determined by Dynamic Light Scattering (DLS).

In a preferred embodiment, said aggregated oligonucleotides have an average diameter of 8-14 nm, preferably 9-14 nm, further preferably 10-14 nm, wherein said average diameter is determined by Dynamic Light Scattering (DLS).

In a further preferred embodiment, said aggregated oligonucleotides have an average diameter of 11-13 nm, preferably 11, 12 or 13 nm, further preferably 12 nm, wherein said average diameter is determined by Dynamic Light Scattering (DLS).

In a further preferred embodiment, at least 90%, preferably at least 95%, of said aggregated oligonucleotides have a diameter of 10.8 nm to 13.2 nm, wherein said diameter is determined by Dynamic Light Scattering (DLS).

In a further preferred embodiment, said at least 65%, preferably at least 70% of said aggregated oligonucleotides have a diameter of 11.4 nm to 12.6 nm, wherein said diameter is determined by Dynamic Light Scattering (DLS).

In a further preferred embodiment of said nucleotide composition, at least 90%, preferably at least 95%, of said aggregated oligonucleotides have a diameter of 12 nm +10%, i.e. have a diameter of 10.8 nm to 13.2 nm, wherein said diameter is determined by Dynamic Light Scattering (DLS).

In a further preferred embodiment of said nucleotide composition, wherein at least 65%, preferably at least 70% of said aggregated oligonucleotides have a diameter of 12nm +5%, i.e. have a diameter of 11.4 nm to 12.6 nm, wherein said diameter is determined by Dynamic Light Scattering (DLS).

In a further preferred embodiment, said aggregated oligonucleotides have the nucleic acid sequence G10 GGGGGGGGGGGACGATCGTCGGGGGGGGGG (SEQ ID NO:1).

In a further preferred embodiment, said aggregated oligonucleotides have the nucleic acid sequence G10 GGGGGGGGGGGACGATCGTCGGGGGGGGGG (SEQ ID NO:1), and wherein said aggregated oligonucleotides exclusively consists of phosphodiester connected deoxynucleotides.

In an again further aspect, the present invention provides for a nucleotide composition comprising aggregated oligonucleotides, wherein said aggregated oligonucleotides have an average diameter of 7-14 nm, wherein said average diameter is determined by Dynamic Light Scattering (DLS).

In a preferred embodiment, said aggregated oligonucleotides have an average diameter of 8-14 nm, preferably 9-14 nm, further preferably 10-14 nm, wherein said average diameter is determined by Dynamic Light Scattering (DLS).

In a further preferred embodiment, said aggregated oligonucleotides have an average diameter of 11-13 nm, preferably 11, 12 or 13 nm, further preferably 12 nm, wherein said average diameter is determined by Dynamic Light Scattering (DLS).

In a further preferred embodiment, wherein at least 90%, preferably at least 95%, of said aggregated oligonucleotides have a diameter of 10.8 nm to 13.2 nm, wherein said diameter is determined by Dynamic Light Scattering (DLS).

In a further preferred embodiment, wherein at least 65%, preferably at least 70% of said aggregated oligonucleotides have a diameter of 11.4 nm to 12.6 nm, wherein said diameter is determined by Dynamic Light Scattering (DLS).

In a further preferred embodiment of said nucleotide composition, at least 90%, preferably at least 95%, of said aggregated oligonucleotides have a diameter of 12 nm+10%, i.e. have a diameter of 10.8 nm to 13.2 nm, wherein said diameter is determined by Dynamic Light Scattering (DLS).

In a further preferred embodiment of said nucleotide composition, wherein at least 65%, preferably at least 70% of said aggregated oligonucleotides have a diameter of 12nm +5%, i.e. have a diameter of 11.4 nm to 12.6 nm, wherein said diameter is determined by Dynamic Light Scattering (DLS).

In a further preferred embodiment, said aggregated oligonucleotides have the nucleic acid sequence G10 GGGGGGGGGGGACGATCGTCGGGGGGGGGG (SEQ ID NO:1).

In a further preferred embodiment, said aggregated oligonucleotides have the nucleic acid sequence G10 GGGGGGGGGGGACGATCGTCGGGGGGGGGG (SEQ ID NO:1), and wherein said aggregated oligonucleotides exclusively consists of phosphodiester connected deoxynucleotides.

In a further aspect, the present invention provides for a process for producing a composition comprising (i) a virus-like particle, wherein said virus-like particle is a virus-like particle of an RNA bacteriophage, and (ii) aggregated oligonucleotides, wherein said aggregated oligonucleotides are packaged into said virus-like particle, said process comprising the steps of: (a) generating a mixture, wherein said mixture comprises: (i) a coat protein of said RNA bacteriophage; (ii) an agent capable of preventing the self-assembly of said coat protein; and (iii) aggregated oligonucleotides, wherein said aggregated oligonucleotides comprise oligonucleotides comprising at least one poly G stretch, and wherein said aggregated oligonucleotides have an average diameter of 6-16 nm, wherein preferably said average diameter is determined by Dynamic Light Scattering (DLS); (b) removing said agent from said mixture; and (c) allowing said coat protein to self-assemble into a virus-like particle and to package said aggregated oligonucleotides.

In an again further aspect, the present invention provides for a process for producing a composition comprising (i) a virus-like particle, wherein said virus-like particle is a virus-like particle of an RNA bacteriophage, and (ii) aggregated oligonucleotides, wherein said aggregated oligonucleotides are packaged into said virus-like particle, said process comprising the steps of: (a) generating a mixture, wherein said mixture comprises: (i) a coat protein of said RNA bacteriophage; (ii) an agent capable of preventing the self-assembly of said coat protein; and (iii) aggregated oligonucleotides, wherein said aggregated oligonucleotides comprise oligonucleotides comprising at least one poly G stretch, and wherein said aggregated oligonucleotides are obtainable by the process according to the first aspect of the present invention, and wherein said aggregated oligonucleotides have an average diameter of 6-16 nm, wherein preferably said average diameter is determined by Dynamic Light Scattering (DLS); (b) removing said agent from said mixture; and (c) allowing said coat protein to self-assemble into a virus-like particle and to package said aggregated oligonucleotides.

In an again further aspect, the present invention provides for a process for producing a composition comprising (i) a virus-like particle, wherein said virus-like particle is a virus-like particle of an RNA bacteriophage, and (ii) aggregated oligonucleotides, wherein said aggregated oligonucleotides are packaged into said virus-like particle, said process comprising the steps of: (a) generating a mixture, wherein said mixture comprises: (i) a coat protein of said RNA bacteriophage; (ii) an agent capable of preventing the self-assembly of said coat protein; and (iii) the nucleotide composition, wherein said nucleotide composition is obtainable by the process for producing a nucleotide composition comprising aggregated oligonucleotides in accordance with the present invention, and wherein said nucleotide composition comprises said aggregated oligonucleotides, wherein said aggregated oligonucleotides comprise oligonucleotides comprising at least one poly G stretch, and wherein said aggregated oligonucleotides have an average diameter of 6-16 nm, wherein preferably said average diameter is determined by Dynamic Light Scattering (DLS); (b) removing said agent from said mixture; and (c) allowing said coat protein to self-assemble into a virus-like particle and to package said aggregated oligonucleotides.

During said processes said virus-like particle is formed by self-assembly of coat protein of said RNA bacteriophage in the presence of said aggregated oligonucleotides.

In a preferred embodiment, said aggregated oligonucleotides have an average diameter of 8-14 nm, preferably 9-14 nm, further preferably 10-14 nm, wherein said average diameter is determined by Dynamic Light Scattering (DLS).

In a further preferred embodiment, said aggregated oligonucleotides have an average diameter of 11-13 nm, preferably 11, 12 or 13 nm, further preferably 12 nm, wherein said average diameter is determined by Dynamic Light Scattering (DLS).

In a further preferred embodiment, said coat protein comprises recombinant proteins, or fragments thereof, that are capable of self-assembly, of a RNA bacteriophage.

In a further preferred embodiment, said coat protein consists of recombinant proteins, or fragments thereof, that are capable of self-assembly, of a RNA bacteriophage.

In a further preferred embodiment, said RNA bacteriophage is selected from the group consisting of:
(a) bacteriophage Qβ;
(b) bacteriophage R17;
(c) bacteriophage fr;
(d) bacteriophage GA;
(d) bacteriophage SP;
(e) bacteriophage MS2;
(f) bacteriophage M11;
(g) bacteriophage MX1;
(h) bacteriophage NL95;
(i) bacteriophage f2;
(j) bacteriophage PP7; and
(k) bacteriophage AP205.

In a further preferred embodiment, said RNA bacteriophage is Qβ.

In a further preferred embodiment, said coat protein comprises a sequence selected from the group consisting of:
(a) SEQ ID NO:10 (Qβ CP);
(b) a mixture of SEQ ID NO:10 and SEQ ID NO:11 (Qβ A1 protein);
(c) SEQ ID NO: 12 (R17 coat protein);
(d) SEQ ID NO: 13 (fr coat protein);
(e) SEQ ID NO: 14 (GA coat protein);
(f) SEQ ID NO: 15 (SP coat protein);
(g) a mixture of SEQ ID NO:15 and SEQ ID NO:16;
(h) SEQ ID NO:17 (MS2 coat protein);
(i) SEQ ID NO:18 (M11 coat protein);
(j) SEQ ID NO:19 (MXI coat protein);
(k) SEQ ID NO:20 (NL95 coat protein);
(l) SEQ ID NO:21 (f2 coat protein);
(m) SEQ ID NO:22 (PP7 coat protein); and
(n) SEQ ID NO:23 (AP205 coat protein).

In a further preferred embodiment, said coat protein comprises the sequence of SEQ ID NO:10 (Qβ CP).

In a further preferred embodiment, said coat protein comprises a mixture of SEQ ID NO:10 and SEQ ID NO:11 (Qβ A1 protein).

In a further preferred embodiment, said coat protein consist of the sequence of SEQ ID NO:10 (Qβ CP).

In a further preferred embodiment, said coat protein consists of a mixture of SEQ ID NO:10 and SEQ ID NO:11 (Qβ A1 protein).

In a further preferred embodiment, the concentration of said coat protein in said mixture is 1 to 4 mg/ml, preferably 2.5 mg/ml.

In a further preferred embodiment, the concentration of said aggregated oligonucleotides in said mixture is 25 to 100 μM, preferably 62.5 μM.

In a further preferred embodiment, said the molar ratio of said aggregated oligonucleotides and said coat protein in said mixture is 0.5 to 1.2, preferably 0.7.

In a further preferred embodiment, said agent comprises a denaturing compound selected from urea and guanidinium hydrochloride.

In a further preferred embodiment, said agent comprises a denaturing compound, wherein said denaturing compound is urea, and wherein preferably the concentration of said urea in said mixture is 0.25 to 7.2 M, preferably 1 M.

In a further preferred embodiment, said agent further comprises a reducing agent.

In a further preferred embodiment, said reducing agent is DTT, wherein preferably the concentration of said DTT in said mixture is 1 to 25 mM, preferably 2.5 mM.

In a further preferred embodiment, said removing of said agent from said mixture is performed by a first buffer exchange with a first buffer, wherein said first buffer comprises sodium chloride, and wherein preferably the concentration of said sodium chloride in said first buffer is 50 to 350 mM, preferably 250 mM.

In a further preferred embodiment, said first buffer exchange is performed across a membrane, wherein said membrane comprises a molecular weight cut off of 1 to 50 kD, preferably of 5 to 30 kD, most preferably of 30 kD.

In a further preferred embodiment, said process further comprises the step of contacting said virus-like particle with an oxidizing agent, wherein preferably said oxidizing agent is selected from the group consisting of
(a) hydrogen peroxide, wherein preferably the concentration of said hydrogen peroxide is 0.25-50 mM, preferably 2 mM;
(b) oxygen;
(c) gluthathion;
(d) Cu²⁺; and
(c) Fe³⁺.

In a further preferred embodiment, said oxygen as oxidizing agent can be sterile filtered air, typically and preferably sterile filtered ambient air.

In a further preferred embodiment, said process further comprises the step of purifying said virus-like particle, and wherein said purifying comprises a second buffer exchange with a second buffer, wherein said second buffer is a pharmaceutically acceptable buffer.

In a further preferred embodiment, said second buffer exchange is performed using a membrane, wherein said membrane comprises a molecular weight cut off of 50 to 1000 kD.

In a further preferred embodiment, said second buffer exchange is performed using a membrane, wherein said membrane comprises a molecular weight cut off of 100 to 300 kD.

In a further preferred embodiment, said the purity of said composition is at least 99.5%, preferably at least 99.6%, more preferably at least 99.7%, still more preferably at least 99.8%, and most preferably at least 99.9% as determined by size exclusion chromatography.

In an again further aspect, the present invention provides for a composition comprising (i) a virus-like particle of an RNA bacteriophage, and (ii) aggregated oligonucleotides, wherein said aggregated oligonucleotides are packaged into said virus-like particle, wherein said composition is obtainable by a process for producing a composition comprising (i) a virus-like particle, wherein said virus-like particle is a virus-like particle of an RNA bacteriophage, and (ii) aggregated oligonucleotides in accordance with the present invention.

In an again further aspect, the present invention provides for a composition comprising (i) a virus-like particle of an RNA bacteriophage, and (ii) aggregated oligonucleotides, wherein said aggregated oligonucleotides are packaged into said virus-like particle, wherein said aggregated oligonucleotides have an average diameter of 6-16, preferably of 7-14 nm, wherein said average diameter is determined by Dynamic Light Scattering (DLS).

In a further preferred embodiment, said RNA bacteriophage is bacteriophage Qβ.

In a further preferred embodiment, said virus-like particle of RNA bacteriophage Qβ consists of coat proteins comprising the sequence of SEQ ID NO:10 (Qβ CP).

In a further preferred embodiment, said virus-like particle of RNA bacteriophage Qβ consists of coat proteins comprising a mixture of SEQ ID NO:10 and SEQ ID NO:11 (Qβ A1 protein).

In a further preferred embodiment, said virus-like particle of RNA bacteriophage Qβ consists of coat proteins consisting of the sequence of SEQ ID NO:10 (Qβ CP).

In a further preferred embodiment, said virus-like particle of RNA bacteriophage Qβ consists of coat proteins consisting of a mixture of SEQ ID NO:10 and SEQ ID NO:11 (Qβ A1 protein).

In a further preferred embodiment, said aggregated oligonucleotides have the nucleic acid sequence G10 GGGGGGGGGG GACGATCGTC GGGGGGGGGG (SEQ ID NO:1).

In a further preferred embodiment, said aggregated oligonucleotides have the nucleic acid sequence G10 GGGGGGGGGGGACGATCGTCGGGGGGGGGG (SEQ ID NO:1), and wherein said aggregated oligonucleotides exclusively consists of phosphodiester connected deoxynucleotides.

In a further preferred embodiment, the purity of said composition is at least 99.5%, preferably at least 99.6%, more preferably at least 99.7%, still more preferably at least 99.8%, and most preferably at least 99.9% as determined by size exclusion chromatography.

In a further preferred embodiment, said aggregated oligonucleotides have an average diameter of 6-16, preferably of 7-14 nm, wherein said average diameter is determined by Dynamic Light Scattering (DLS).

In a further preferred embodiment, said aggregated oligonucleotides have an average diameter of 8-14 nm, preferably 9-14 nm, further preferably 10-14 nm, wherein said average diameter is determined by Dynamic Light Scattering (DLS).

In a further preferred embodiment, said aggregated oligonucleotides have an average diameter of 11-13 nm, preferably 11, 12 or 13 nm, further preferably 12 nm, wherein said average diameter is determined by Dynamic Light Scattering (DLS).

In a further preferred embodiment, wherein at least 90%, preferably at least 95%, of said aggregated oligonucleotides have a diameter of 10.8 nm to 13.2 nm, wherein said diameter is determined by Dynamic Light Scattering (DLS).

In a further preferred embodiment, wherein at least 65%, preferably at least 70% of said aggregated oligonucleotides have a diameter of 11.4 nm to 12.6 nm, wherein said diameter is determined by Dynamic Light Scattering (DLS).

In a further preferred embodiment of said composition, at least 90%, preferably at least 95%, of said aggregated oligonucleotides have a diameter of 12 nm±10%, i.e. have a diameter of 10.8 nm to 13.2 nm, wherein said diameter is determined by Dynamic Light Scattering (DLS).

In a further preferred embodiment of said composition, wherein at least 65%, preferably at least 70% of said aggregated oligonucleotides have a diameter of 12nm±5%, i.e. have a diameter of 11.4 nm to 12.6 nm, wherein said diameter is determined by Dynamic Light Scattering (DLS).

### EXAMPLES

The Examples are intended to illustrate the present invention without restricting it. In the Examples described below, unless otherwise indicated, all temperatures are set forth in degrees Celsius (° C.). Reagents were purchased from commercial suppliers such as Sigma Aldrich, Boston Bioproducts, Invitrogen, Alfa Aesar or the like, and were used without further purification unless otherwise indicated. The water used in the described reactions has been purified or treated to remove all contaminants and salts. The removal of inorganic ionic impurities is confirmed by measuring the conductivity of the water. Water used in this application has a resistivity of typically and preferably at least 18 MΩ·cm at 25° C. This ensures residual inorganic impurities such as salts are less than 1 ppb.

The purity of the oligonucleotides, in particular, of the oligonucleotide G10 of SEQ ID NO:1, was determined by ion-pair, reversed-phase high performance liquid chromatography (IP-RP-HPLC) or by anion exchange—high performance liquid chromatography (IEX-HPLC).

IP-RP-HPLC was effected using a Waters Xbridge BEH C18 4.6×75 mm, 2.5 μm column at a column temperature of 70±2° C., a flow rate of 0.4 mL/min, a wavelength of 260 nm, an injection volume of 5 μL and a run time of 40 minutes.

IEX-HPLC was effected using a Dionex DNAPac PA200 4.0 x 250 mm part #063000 column at a column temperature of 30±2° C., a flow rate of 1.0 mL/min, a wavelength of 260nm, an injection volume of 20 μL and a run time of 45 minutes.

IP-RP-HPL.C//G10: Samples are injected on a oligonucleotide ion pairing column and elution is carried out using a combined water acetonitrile gradient modified with TEA and HFIP as the ion pairing buffer with detection at 260 nm. The resulting oligonucleotide G10 peak is integrated separately from the remaining peaks consisting of oligonucleotide G10 subpopulations such as G10+1n, G10−1n, G10−2n, G10−3n, >G10+1n and <G10−3n (whereby n=deoxynucleotide).

IEX-HPLC//G10: Samples are injected on a strong anion-exchange column and analyzed under denaturing conditions (pH>10). Elution is carried out using a combined salt and methanol gradient with detection at 260 nm. The resulting oligonucleotide G10 peak is integrated separately from the remaining peaks consisting of oligonucleotide G10 subpopulations such as G10+1n, G10−1n, G10−2n, G10−3n, >G10+1n and <G10−3n (whereby n=deoxynucleotide).

### Example 1

### Denaturation and Aggregation of Oligonucleotide G10 (SEQ ID NO:1)

Quantification of G10: Oligonucleotide G10 (SEQ ID NO:1) was quantified by UV absorption at 260 nm corrected by the absorption at 340 nm, wherein 1 A₂₆₀₋₃₄₀ corresponds to a concentration of 27.8 μg/ml at 1 cm path length.

Denaturation: (10.0 ml scale, 500 μM G10 of a purity of about 94% as determined by reverse phase HPLC and anion Exchange HPLC (referred to as high purity G10 oligonucleotide in this Example section), 1M Urea, 85° C., 20 min): 70.6 mg G10 were weighed into a 15 ml tube. The powder was dissolved in 10.0 ml purified water (with a resistivity at 25° C. of 18.2 MΩ·cm) containing 1M urea (c=500 μM; content of bulk powder determined prior to dilution by spectrometry). The mixture was disaggregated for 20 minutes at 85° C. in a water bath. Aliquots were taken, immediately cooled down in an ice/water bath to 0° C., removed from the ice bath, and allowed to warm to room temperature naturally, and DLS and optionally size exclusion HPLC (SEC) measurements conducted as described in Example 3 and Example 4. The remainder of the sample was held at 85° C. and the aggregation step conducted.

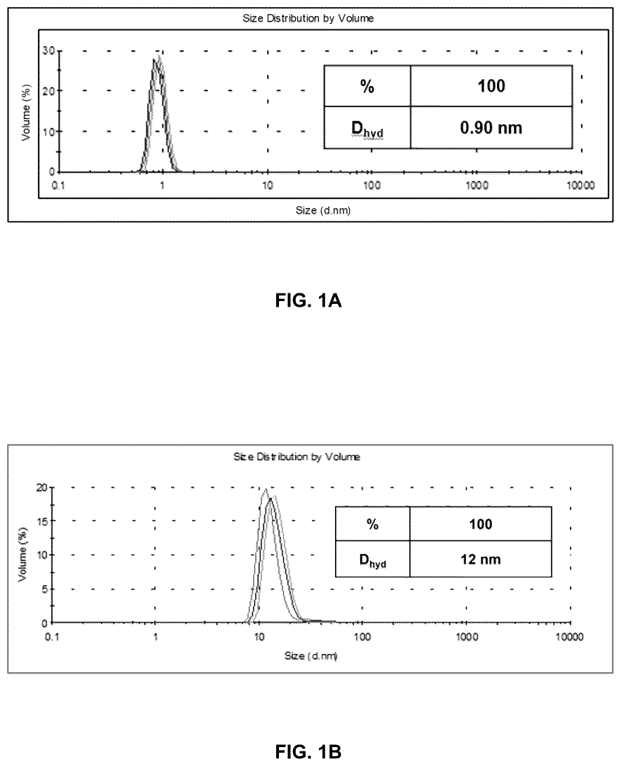

FIG. 1A shows a DLS of the denatured high purity G10 oligonucleotide obtained with the inventive process as a single peak. The measured average diameter of the G10 oligonucleotide of 0.90 nm indicates that denaturing is complete and monomers have been achieved. This fully denatured G10 oligonucleotide ensures and allows the formation a well-controlled and defined aggregated G10 oligonucleotides within the desired range in accordance with the present invention as outlined below.

Aggregation (20.0 ml scale, 250 μM G10 denatured as above, 250 mM Na⁺Cl⁻ 20 mM Sodium Phosphate (pH=7.2), 1M urea 85° C., 8-30 min): 10 ml of the denatured 500 M G10 solution at 85° C. referred above were mixed with a solution of 10 ml of 500 mM NaCl, 40 mM Sodium Phosphate, and 1M urea at 85° C. in a 25 ml tube. The mixture was incubated for 15 minutes at 85° C. in a water bath. The solution was cooled down in an ice/water bath to 0° C. Aliquots were taken therefrom, allowed to warm to room temperature, and DLS and optionally size exclusion HPLC (SEC) measurements conducted. Aggregated oligonucleotide solutions are typically and preferably used within 3 hours if stored <20° C.

A final filtration step can be utilized to remove eventual trace (<1%) of large particles. This is performed by passing the previously cooled, room temperature solution of aggregated oligonucleotides through a 50 nm filter.

FIG. 1B shows a DLS of the subsequently aggregated G10 oligonucleotides obtained with the inventive process. The very preferred aggregated G10 oligonucleotides obtained show proper aggregation and an average diameter of 12 nm. These well-controlled and defined aggregated G10 oligonucleotides within the desired preferred range in accordance with the present invention will result in very high purity packaged and well-formed VLPs as outlined below.

The denaturation and aggregation of the very preferred oligonucleotide G10 (SEQ ID NO:1) were further conducted at different oligonucleotide concentrations, wherein basically the same DLS as depicted in FIG. 1A and FIG. 1B were obtained. Thus, denaturation was effected using various concentrations of the oligonucleotide G10 of between 100 μm and 1 mM. The subsequent aggregation step were then effected by exactly half that concentration as used for the denaturation as a matter of convenience due to a 1:1 mixing of the two solutions as described herein.

### Example 2

### Denaturation and Aggregation of Oligonucleotides G10-11, G12-11, G6, G7, G8, G9, G11, G6-10, G7-10, G8-10, and G9-10 (SEQ ID NOs: 3-9, 24-27)

Denaturation: A solution of 500 μM oligonucleotide G10-11 (SEQ ID NO:3), G12-11 (SEQ ID NO:4), G6 (SEQ ID NO:5), G7 (SEQ ID NO:6), G8 (SEQ ID NO:7), G9 (SEQ ID NO:8), G11 (SEQ ID NO:9), G6-10 (SEQ ID NO:24), G7-10 (SEQ ID NO:25), G8-10 (SEQ ID NO:26) or G9-10 (SEQ ID NO:27) in IM urea was disaggregated for 20 minutes at 85° C. in a water bath.

Aggregation: (10.0 ml scale, 250 μM G10, 250 mM Na⁺, 20 mM Sodium Phosphate, 1M urea 85° C., 8-30 min): 5 ml denatured solution of oligonucleotides G10-11 (SEQ ID NO:3), G12-11 (SEQ ID NO:4), G6 (SEQ ID NO:5), G7 (SEQ ID NO:6), G8 (SEQ ID NO:7), G9 (SEQ ID NO:8), G11 (SEQ ID NO:9), G6-10 (SEQ ID NO:24), G7-10 (SEQ ID NO:25), G8-10 (SEQ ID NO:26) or G9-10 (SEQ ID NO:27) at 85° C., 5 ml of 500 mM Na⁺, 40 mM Sodium Phosphate, and 1M urea at 85° C. were mixed in a 15 ml tube (250 μM oligo, 1 M urea, 20 mM Sodium Phosphate, 250 mM Na⁺). The mixture was incubated for 15 minutes at 85° C. in a water bath. The solution was cooled down in an ice/water bath to 0° C. Aliquots were taken therefrom, allowed to warm to room temperature, and DLS and optionally size exclusion HPLC (SEC) measurements conducted. Aggregated oligonucleotide solutions are typically and preferably used within 3 hours if stored <20° C.

The products of the aggregation processes were analyzed by Dynamic Light Scattering (DLS) as described in Example 3 and by size exclusion HPLC as described in Example 4. DLS of the aggregated oligonucleotides revealed that the average diameter of all the aggregated oligonucleotides were between 11-13 nm, as determined by Dynamic Light Scattering (DLS), and all within 80%-110% PST, as determined by size exclusion HPLC.

### Example 3

### Analysis of the Aggregation of Oligonucleotide G10 by Dynamic Light Scattering

The particle size of the oligonucleotides, aggregated oligonucleotides and VLPs packaged with aggregated oligonucleotides in accordance with the present invention was determined using dynamic light scattering (DLS). Instrument settings as used for the present examples and as preferred for determining the particle size of the oligonucleotides, aggregated oligonucleotides and VLPs packaged with aggregated oligonucleotides in accordance with the present invention are presented below.

Instrument: Malvern Zetasizer Nano ZS

Light Source: He—Ne laser (633 nm at 4 mW (maximum))

The middle column of Table 1 represents the settings applied for a reference standard (polystyrene microspheres) and for calibration that confirmed that the instrument was performing properly. The right column represents the settings applied in the method used for our analysis and measurements of the oligonucleotides, aggregated oligonucleotides and VLPs packaged with aggregated oligonucleotides.

TABLE 1
Instrument settings for standard and inventive compositions

| | | Inventive products, |
|---|---|---|
| | | aggregated |
| | Reference Standard: | oligonucleotides, |
| | polystyrene | compositions of |
| Application: | microspheres | packaged VLPs |
| Type: | Size | Size |
| Material: | Polystyrene Latex | DNA/Protein |
| Refractive Index RI: | 1.59 | 1.45 |
| Absorbance (ABS): | 0.01 | 0 |
| Dispersant: | H₂O | PBS |
| Temperature: | 25° C. | 25° C. |
| Viscosity: | 0.8872 cP | 1.33 cP |
| Refractive Index RI: | 1.33 | 1.33 |
| Equilibration time: | 20 sec | 60 sec |
| Detection angle: | 173° (backscatter) | 173° (backscatter) |
| Run duration: | 10 sec | 30 sec |
| Number of runs per | 3-5, preferably 3 | 3-5, preferably 3 |
| measurement: | | |

The DLS software calculates average hydrodynamic radii, and through basic multiplication, the particle average diameter is determined. 2 ×particle average radius=Particle diameter. For consistency, particle size will henceforth be reported and used throughout the present invention as average diameter measured in nanometers (D_{Hyd}).

### Example 4

### Analysis of the Aggregation of Oligonucleotide G10 by Size Exclusion HPLC

The aggregation state of the aggregated G10 oligonucleotides was analyzed essentially as described in WO 2007/144150 by analytical size exclusion HPLC using the following conditions:

| | |
|---|---|
| Column: | TSKgel 5000 PWXL 7.8 mm * 30.0 cm |
| | (Lot: 5PWX06GNMH3304, Art: 08023, |
| | Tosoh Bioscience) |
| Eluent: | PBS (150 mM NaCl in 20 mM sodium phosphate |
| | buffer, pH 7.2) |
| Injection volume: | 40.0 μl (preferably comprising a concentration |
| | of about 20 μM to about 500 μM) |
| Flow rate: | 0.8 ml/min |
| Gradient: | Isocratic |
| Run time: | 20 min |
| Wavelength: | 215, 260 and 280 nm, data evaluation at 260 nm |
| Column oven temp.: | 25° C. |
| Autosampler temp.: | 8° C. |

Capsid of bacteriophage Qβ was used as standard.

The peak start time X % of G10 relative to Qβ capsid (relative peak start time Qß) was calculated as follows: X %=peak start time [min] of the oligonucleotide divided by the retention time of Qβ capsid standard [min]×100%, wherein the peak start time of the oligonucleotide was determined as the time when the elution of the oligonucleotide became detectable, and wherein the retention time of the Qβ capsid standard was determined as the time of the occurrence of the maximum peak of the standard. An Example of an elution profile of oligonucleotide G10 and capsid of bacteriophage Qβ as standard is depicted in FIG. 1 of WO2007/144150. Based on the chromatograms depicted in FIG. 1 of WO2007/144150 a relative peak start time of 88% was calculated for the aggregated oligonucleotide.

### Example 5

### Comparison of Denatured Oligonucleotides G10 Obtained by the Inventive Process and by Prior Art Processes

The oligonucleotide G10 of SEQ ID NO:1 with two different purities (79% and 94% as determined by reverse phase HPLC and anion Exchange HPLC; referred to as low and high purity G10 oligonucleotide in this Example section) were each subjected to the disaggregation (denaturation) as described in the prior art (WO2007/144150). The same high purity (94%) oligonucleotide G10 was subjected to the denaturation as described in Example 1 herein. The impurities of the used oligonucleotide G10 are mostly “failure sequences” meaning oligonucleotide sequences with lower number of G residues, be it 26 mers, 27 mers, 28 mers and 29 mers.

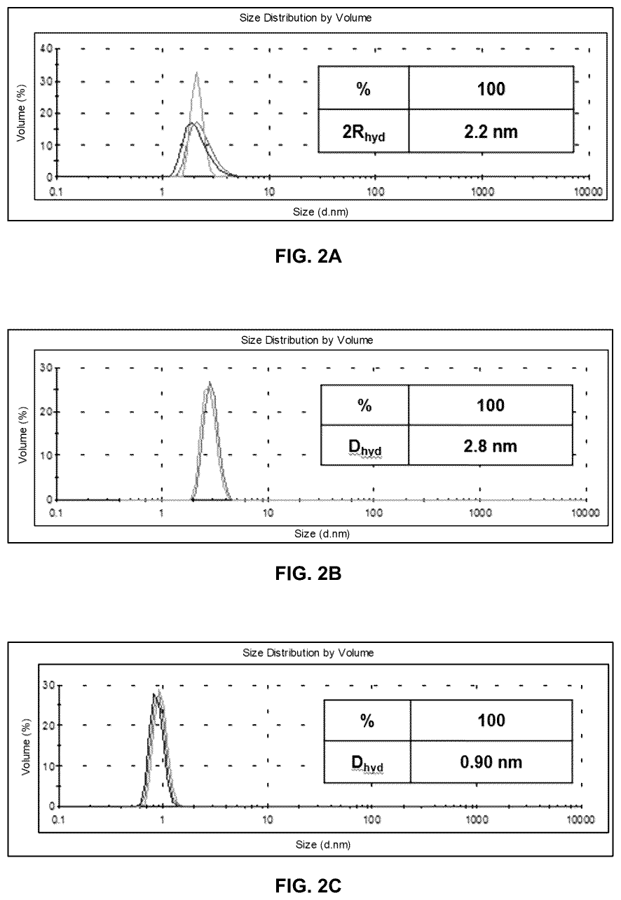

The resulting products were analyzed by DLS as described in Example 3 and are shown in FIG. 2A (Low purity G10, prior art process) and FIG. 2B (High purity G10, prior art process) and FIG. 2C (High purity G10, inventive process). The completely denatured oligonucleotide G10 monomer has a hydrodynamic average diameter of approximately 1 nm.

The low purity G10 subjected to the denaturing prior art process resulted in particles with an average diameter of 2.2 nm indicating the presence of secondary structures and that not all oligonucleotide G10 has been completely denatured to monomer (FIG. 2A). The high purity G10 subjected to the denaturing prior art process resulted in particles with an average diameter of 2.8 nm indicating the presence of secondary structures and that not all oligonucleotide G10 has been completely denatured to monomer (FIG. 2B). As will be discussed in Example 6, this incomplete denaturing will lead to more variable, and further to larger, aggregated oligonucleotides.

The high purity G10 subjected to the denaturing of the inventive process resulted in particles with an average diameter of 0.9 nm indicating that oligonucleotide G10 has been completely or substantially completely denatured to monomer (FIG. 2C). As indicated, this fully denatured G10 oligonucleotide ensures and allows the formation of well-controlled and defined aggregated G10 oligonucleotides within the desired range in accordance with the present invention.

The proper denaturing of the oligonucleotides such as shown for the very preferred oligonucleotide G10 leading to complete, or at least almost complete, denaturing and to monomers prior to the start of the aggregation step is highly preferred and important. If secondary structures exist, dimers, trimers, or quadruplexes of the oligonucleotides, the aggregation step will be more variable, and the final aggregated oligonucleotides will be larger and of a broader size distribution.

### Example 6

### Comparison of Aggregated Oligonucleotides G10 Obtained by the Inventive Process and by Prior Art Processes

The G10 materials obtained from the denaturing experiments as described in Example 5 were subjected to aggregation, either as described in the prior art (WO2007/144150), or as described by the inventive process, and hereby as described in Example 1 above.

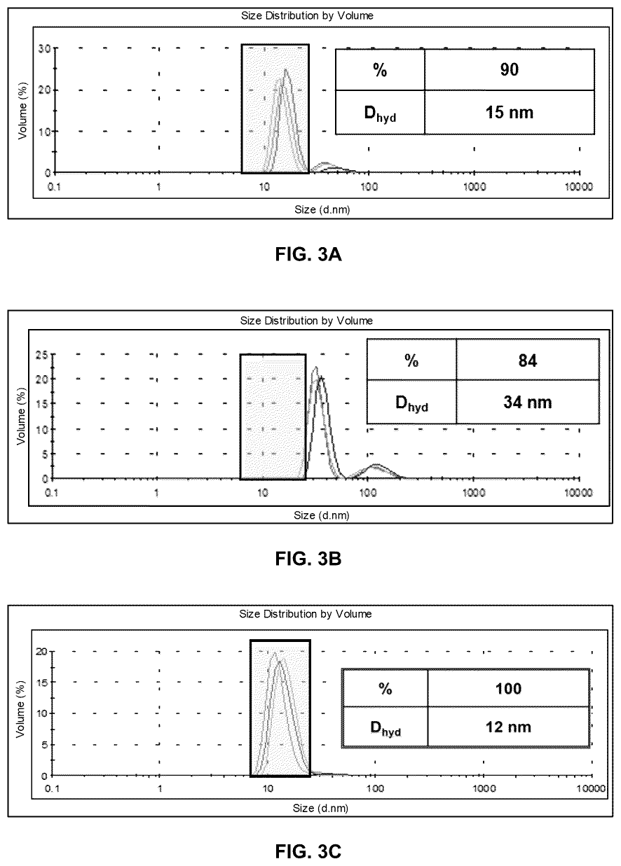

The resulting aggregated oligonucleotides were analyzed by DLS as described in Example 3 herein and are shown in FIG. 3A (prior art aggregation of low purity G10 denatured by prior art process) and FIG. 3B (prior art aggregation of high purity G10 denatured by prior art process) and FIG. 3C (inventive aggregation of high purity G10 denatured by inventive process).

The low purity material denatured and aggregated with the prior art process resulted in aggregated oligonucleotides which are not only on the high side of the desired average diameter range (6-16 nm), but additionally, 10% of the material is too large (30-40 nm) for subsequent proper packaging into VLP's of RNA bacteriophages, preferably of RNA bacteriophages Qβ (FIG. 3A). As a result of this larger and wide particle distribution, the final packaged VLPs will be less pure by SEC and DLS and rod like structures will be observed in the electron micrographs (see Example 8 below). It has to be noted that said rod like structures cannot typically be separated by purification via filtration but requires more costly and intense purification via chromatography which is very detrimental for manufacturing at scale, and in particular, for GMP manufacturing.

The high purity material denatured and aggregated with the prior art process resulted in aggregated oligonucleotides all of which are too large for packaging into VLP's of RNA bacteriophages, preferably of RNA bacteriophages Qβ, and, therefore will result in unstable VLPs (FIG. 3B). Additionally, a second peak at ˜100 nm is identified. It is noteworthy that optimization of the prior art process using the high purity material by decreasing the time of aggregation was not performed due to the fact that the then required heating and cooling times would be shorter than what could be easily controlled at either the lab or at manufacturing scale. The present inventive processes were, in fact, able to overcome said disadvantage of the prior art processes.

Thus, and to the contrary, the high purity material denatured and aggregated with the process of the present invention resulted in aggregated oligonucleotides having an average diameter of 12 nm indicating proper aggregation (FIG. 3B). This well-controlled and defined aggregated G10 oligonucleotides within the desired very preferred range in accordance with the present invention will result in very high purity and well-formed packaged VLPs.

The proper aggregation of the oligonucleotides such as shown for the very preferred oligonucleotide G10 leading to a complete, or at least almost complete, narrowly defined diameter size distribution is further highly preferred and important. Controlling the aggregation leading to aggregated oligonucleotides of an average diameter of 11-13 nm, preferably of an average diameter of 12 nm, as determined by DLS as described in Example 3 ensures and allows to achieve a high purity packaged VLP.

If the aggregated oligonucleotides are too big, the resulting material after the packaging step will have large impurities as shown in the DLS, and malformed VLPs such as rod like structures as shown in the electron micrographs. If the aggregated oligonucleotides are very large >50 nm, unstable VLPs can result.

### Example 7

Packaging of Qβ VLPs with Aggregated Oligonucleotides G10 by Disassembly/Reassembly

Disassembly of Qβ VLPs: 45 mg Qβ VLP (2.5 mg/ml, as determined by Bradford analysis) in PBS (20 mM Phosphate, 150 mM NaCl, pH 7.5), was reduced with 10 mM DTT for 15 min at RT under stirring conditions. Then, magnesium chloride was added to 0.7 M final concentration and the incubation was continued for 15 min at RT under stirring conditions, leading to precipitation of the encapsulated host cell RNA and concomitant disintegration of the VLPs. The solution was centrifuged 10 min at 4000 rpm at 4° C. (Eppendorf 5810 R, in fixed angle rotor A-4-62 used in all following steps) in order to remove the precipitated RNA from the solution. The supernatant, containing the released, dimeric Qβ coat protein, was used for the chromatographic purification steps.

In an alternative and preferred manner, the Qbeta capsid was disassembled into Qbeta dimer by the addition of 1 M DTT to a final concentration of 10 mM DTT. Nucleic acid and host cell proteins were precipitated by increasing the NaCl concentration to 600 mM and adjusting the pH to pH 2.6 by the addition of 1 M Sodium Phosphate, 0.75 M Citric Acid. The precipitated nucleic acids and HCP were removed by TFF using a Sartoflow Beta Crossflow system fitted with 2×0.5 m² Millipore Biomax 300 membranes ran using the following operating parameters: P_{Feed}=0.9, P_{Retentate}=0.4 bar and P_{Permeate}=0.2 bar resulting in a TMP of 0.45 bar. The material was diafiltered against 3 DVs of 20 mM Sodium Phosphate, 20 mM Citric acid, 300 mM Sodium Chloride pH 3.3.

Purification of Qβ coat protein by cation exchange chromatography and size exclusion chromatography: The supernatant of the disassembly reaction, containing dimeric coat protein, host cell proteins and residual host cell RNA, was loaded onto a SP-Sepharose FF column (xk16/20, 6 ml, Amersham Bioscience). The column was equilibrated with 20 mM sodium phosphate buffer pH 7 and the sample was diluted 1:15 in water to adjust a conductivity below 10 mS/cm in order to achieve proper binding of the coat protein to the column. The elution of the bound coat protein was accomplished by a step gradient to 20 mM sodium phosphate/500 mM sodium chloride and the protein was collected in a fraction volume of approx. 25 ml. The chromatography was carried out at RT with a flow rate of 5 ml/min during all steps and the absorbance was monitored at 260 nm and 280° nm. In a second step, the isolated Qβ coat protein (the eluted fraction from the cation exchange column) was loaded onto a Sephacryl S-100 HR column (xk26/60, 320 ml, Amersham Bioscience) equilibrated with 20 mM sodium phosphate/250 mM sodium chloride; pH 7.2. The chromatography was carried out at RT with a flow rate of 2.5 ml/min and the absorbance was monitored at 260 nm and 280 nm. Fractions of 5 ml were collected.

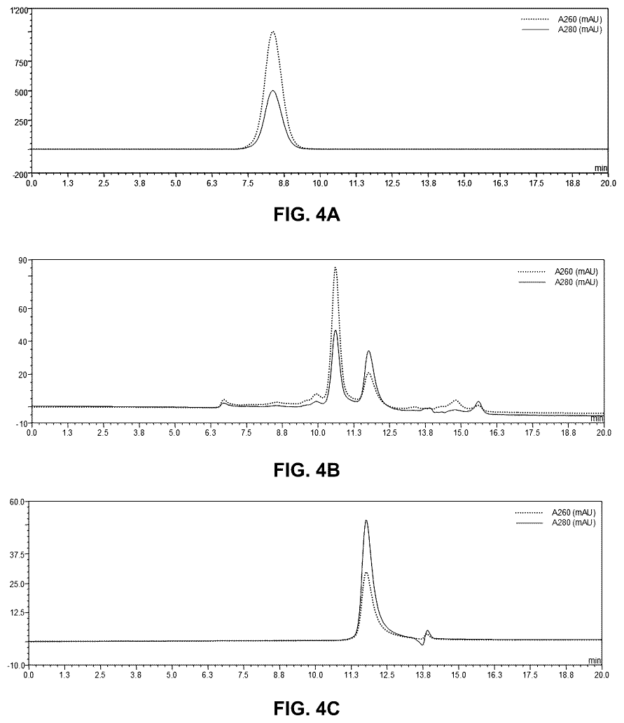

Characterization of purified Qβ coat protein by analytical size exclusion chromatography: A sample of purified Qβ coat protein was analyzed by analytical size exclusion chromatography (FIG. 4C) and compared to i) intact Qβ VLP (FIG. 4A), which had been purified from E. coli lysate and which was used as source material for the purification procedure, and ii) to the supernatant of the disassembly reaction (FIG. 4B). Efficient separation of RNA molecules from the coat protein is indicated by the absence of any RNA-like peak (typical ratio of A280/A260 =0.5) in FIG. 4C and the presence of a unique protein-like peak (typical ratio of A280/A260 =1.7).

In an alternative and preferred manner, purification of Qβ coat protein were effected by cation exchange chromatography and Mustang Q membrane: CEX Chromatography was performed as a capture step for the Qbeta dimer. SP Sepharose FF resin was packed into a BPG140 column using an AKTA Ready Chromatography system and 150 mM NaCl as the packing buffer. The bed height of the packed column was 14.0 cm, equivalent to a bed volume of 2.2 L. HETP analysis gave an asymmetry factor of 1.55 and a theoretical plate count of 2560 plates per meter. The diafiltrate from the disassembly step was filtered through a Millipore Opticap XL 5 capsule prior to loading. Chromatography was performed using the method shown in Table 2.

TABLE 2
Cation Exchange Chromatography Method

| Step | Buffer | CV |
|---|---|---|
| Sanitisation | 0.5M NaOH (5 hr contact time) | 5 |
| Equilibration | 20 mM Sodium Phosphate 300 mM NaCl pH 3.3 | 5 |
| Load | CEX Load | Up to 15 g/L |
| Wash 1 | 20 mM Sodium Phosphate, 300 mM NaCl pH 3.3 | 5 |
| Wash 2 | 20 mM Sodium Phosphate, 300 mM NaCl pH 7.2 | 10 |
| Elution | 20 mM Sodium Phosphate, 550 mM NaCl pH 7.2 | 5 |
| High Salt Regeneration | 20 mM Sodium Phosphate, 1150 mM NaCl pH 7.2 | 3 |
| CIP | 0.5M NaOH (5 hr contact time) | 3 |
| Storage | 20% (v/v) Ethanol, 0.1M NaCl | 1.5 |

A flow rate 144 cm/h for Sanitisation and Equilibration. 216 cm/h was used for all other steps.

Filtration through a Mustang Q capsule was performed to reduce endotoxin and any residual nucleic acid. The CEX Pool was initially filtered using a 0.2 μm Millipak 60 filter (Cat. No. MPGL06GH2) prior to filtration through a Mustang Q filter at a flow rate of 200 ml/min. The flowthrough collected from the Mustang Q filter was then passed through a second 0.2 μm Millipak 60 filter.

Assembly of QβG10 by diafiltration: Purified coat protein (in 20 mM sodium phosphate pH 7.2, 250 mM NaCl) was mixed with water and stock solutions of urea, NaCl, DTT and aggregated G10 oligonucleotides (prepared as described in Example 1). The volume of the mixture was 50 ml and the final concentrations of the components were 1 mg/ml coat protein, 1.0 M urea, 250 mM NaCl, 2.5 mM DTT and 0.24 mg/ml G10. The solution was then diafiltrated at room temperature against 300 ml of 20 mM sodium phosphate 250 mM NaCl pH 7.2, μsing a 30 kDa cut off cartridge (Pellicon XL, Millipore) and a cross flow rate of 10 ml/min and a permeate flow rate of 2.5 ml/min. H₂O₂ was added to 7 mM final concentration and the solution incubated for 1 h at RT in order to induce the formation of disulfide bonds. The solution was then diafiltrated against 500 ml of 20 mM sodium phosphate 150 mM NaCl pH 7.2, μsing a 300 kDa cut off cartridge (Pellicon XL, Millipore) and a cross flow rate of 10 ml/min and a permeate flow rate of 2.5 ml/min, in order to remove excess of H₂O₂ and non-packaged G10 oligonucleotides from the assembled QβG10 product.

Alternatively, the packaging of Qβ VLPs with the aggregated oligonucleotides G10 as obtained by the present invention can also further be effected as described in Example 10 of WO2007/144150.

### Example 8

### Comparison of Qβ VLPs Packaged With Aggregated Oligonucleotides G10 as Obtained by the Present Invention and by Prior Art Processes-DLS and EM

Qβ VLPs were prepared in analogy to Example 7 described above using not only the aggregated G10 oligonucleotides prepared as described in Example 1, but further using the aggregated G10 oligonucleotides prepared by the prior art processes as described in Example 6 above.

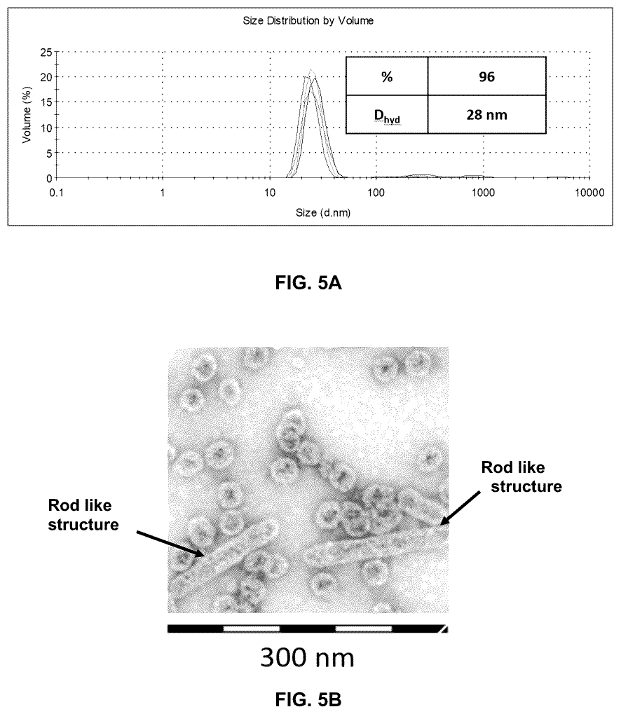

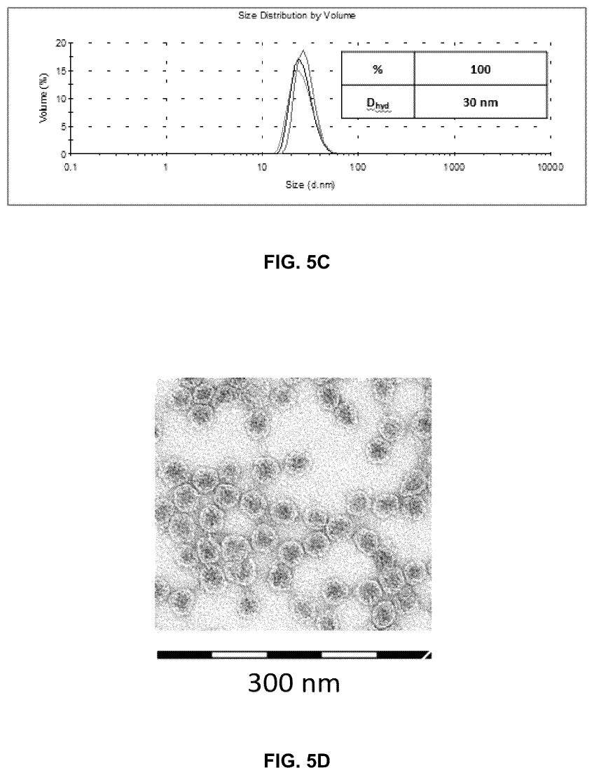

The aggregated oligonucleotides shown in FIG. 3A, which were obtained by prior art aggregation of low purity G10 denatured by prior art process, and having a wide size distribution with 10% being too large for packaging, when subjected to the packaging step resulted in VLPs having a DLS as shown in FIG. 5A and an EM as shown in FIG. 5B. The DLS revealed a major peak (96%) of an average diameter of 28 nm corresponding to properly formed VLPs (30 nm±2 nm), but additional large particle peaks were observed as well. The corresponding EM shows spherical VLPs with said average diameter, but also rod like structures much larger than the desired 30 nm VLPs.

To the contrary, the high purity material denatured and aggregated with the process of the present invention when subjected to the packaging step resulted in packaged VLP's of one average diameter and purely formed VLPs. The DLS shows one single peak at 30 nm and no large particles (FIG. 5C) and the EM shows all spherical VLPs with no rod like structures (FIG. 5D).

### Example 9

### Denaturing Step of the Inventive Process Effected With Varied Parameters

The denaturation of oligonucleotide G10 (SEQ ID NO:1) as described in Example 1 was investigated by varying the concentration of urea, denaturing time, and temperature applied for said denaturation. A bulk oligonucleotide G10 solution was obtained by dissolving G10 (high purity of 94%) in water to a concentration of 1 mM. Urea solutions were added to obtain final denaturing solutions of 500 μM G10 with urea concentrations from 0.1M to 1M. Aliquots of these samples were then incubated at a range of temperature between 25° C. and 85° C. for 20 or 60 minutes. Samples were immediately cooled down in an ice/water bath to 0° C., removed from the ice bath, and allowed to warm to room temperature naturally, and DLS measurements as described in Example 3 conducted. Table 3 shows that successful denaturing, i.e. an average diameter of 1 nm or less, can be achieved irrespective of the urea concentration of 0.2 M to 1.0 M.

TABLE 3
Urea Concentration and Temperature
Impact on G10 Oligonucleotide Denaturing

| Urea | Time | Temperature | Average Diameter |
|---|---|---|---|
| Concentration | (min) | (° C.) | of 1 nm or less |
| 0.1M | 60 | 85 | No |
| 0.2M | 60 | 85 | Yes |
| 0.5M | 20 | 85 | Yes |
| 1.0M | 60 | 75 | Yes |
| 1.0M | 20 | 85 | Yes |
| 1.0M | 60 | 85 | Yes |

1-26. (canceled)

27. A process for producing a composition comprising
(1) a virus-like particle, wherein said virus-like particle is a virus-like particle of an RNA bacteriophage, and
(2) aggregated oligonucleotides, wherein said aggregated oligonucleotides are packaged into said virus-like particle,
said process comprising the steps of:
(A) generating a mixture, wherein said mixture comprises:
(i) a coat protein of said RNA bacteriophage;
(ii) an agent capable of preventing the self-assembly of said coat protein; and
(iii) aggregated oligonucleotides, wherein said aggregated oligonucleotides comprise oligonucleotides comprising at least one poly G stretch,
(B) removing said agent from said mixture; and
(C) allowing said coat protein to self-assemble into a virus-like particle and to package said aggregated oligonucleotides;
wherein said aggregated oligonucleotides are obtained by a process comprising
(a) providing oligonucleotides which comprise 3 to 15 guanosine entities at the 5′ end and 3 to 15 guanosine entities at the 3′ end, and wherein said oligonucleotides comprise phosphodiester connected deoxynucleotides;
(b) denaturing said oligonucleotides, wherein said denaturing comprises a step of incubating an aqueous solution I comprising said oligonucleotides and a chaotropic agent at 75° C. to 99° C. until the average diameter of said oligonucleotides is 1 nm or less, wherein said average diameter is determined by Dynamic Light Scattering (DLS); and
(c) aggregating said oligonucleotides, wherein said aggregating comprises the steps of
(i) incubating an aqueous solution II comprising said oligonucleotides having said average diameter of 1 nm or less obtained in step (b), a chaotropic agent and a cation at 75° C. to 99° C. to form said aggregated oligonucleotides, wherein said incubating is performed until the average diameter of said formed aggregated oligonucleotides is 6-16 nm, wherein said average diameter is determined by Dynamic Light Scattering (DLS), and
(ii) adjusting the temperature of said solution II to below 40° C.

28. The process of claim 27, wherein said aqueous solution I does not comprise mono or divalent ions in a concentration such that said oligonucleotides spontaneously self-aggregate.

29. The process of claim 27, wherein said chaotropic agent comprised in said solution I is selected from urea, phenol, isopropyl alcohol, ethanol and guanidinium chloride.

30. The process of claim 27, wherein said chaotropic agent comprised in said solution II is selected from urea, phenol, isopropyl alcohol, ethanol and guanidinium chloride.

31. The process of claim 27, wherein said chaotropic agent comprised in said solution I and said chaotropic agent comprised in said solution II is urea.

32. The process of claim 27, wherein said oligonucleotides comprise 10 to 100 nucleotides.

33. The process of claim 27, wherein said aggregated oligonucleotides have the nucleic acid sequence comprise a nucleotide sequence selected from the group consisting of:
(a) G10:
(SEQ ID NO: 1)
GGGGGGGGGGGACGATCGTCGGGGGGGGGG;
(b) G10-11:
(SEQ ID NO: 3)
GGGGGGGGGGGACGATCGTCGGGGGGGGGGG;
(c) G12-11:
(SEQ ID NO: 4)
GGGGGGGGGGGGGACGATCGTCGGGGGGGGGGG
(d) G6:
(SEQ ID NO: 5)
GGGGGGGACGATCGTCGGGGGG;
(e) G7:
(SEQ ID NO: 6)
GGGGGGGGACGATCGTCGGGGGGG;
(f) G8:
(SEQ ID NO: 7)
GGGGGGGGGACGATCGTCGGGGGGGG;
(g) G9:
(SEQ ID NO: 8)
GGGGGGGGGGACGATCGTCGGGGGGGGG;
(h) G11:
(SEQ ID NO: 9)
GGGGGGGGGGGGACGATCGTCGGGGGGGGGGG
(i) G6-10:
(SEQ ID NO: 24)
GGGGGGGACGATCGTCGGGGGGGGGG;
(j) G7-10:
(SEQ ID NO: 25)
GGGGGGGGACGATCGTCGGGGGGGGGG;
(k) G8-10:
(SEQ ID NO: 26)
GGGGGGGGGACGATCGTCGGGGGGGGGG;
and
(l) G9-10:
(SEQ ID NO: 27)
GGGGGGGGGGACGATCGTCGGGGGGGGGG.

34. The process of claim 27, wherein said oligonucleotides have the nucleic acid sequence GGGGGGGGGGGACGATCGTCGGGGGGGGGG (SEQ ID NO:1) (G10).

35. The process of claim 27, wherein the purity of said oligonucleotides in step (a) is 90% or higher, as determined by HPLC.

36. The process of claim 27, wherein at least 90% said aggregated oligonucleotides have an average diameter of 9-14 nm,
wherein said average diameter is determined by Dynamic Light Scattering (DLS).

37. The process of claim 27, wherein said RNA bacteriophage is selected from the group consisting of:
(a) bacteriophage Qβ;
(b) bacteriophage R17;
(c) bacteriophage fr;
(d) bacteriophage GA;
(d) bacteriophage SP;
(e) bacteriophage MS2;
(f) bacteriophage M11;
(g) bacteriophage MX1;
(h) bacteriophage NL95;
(i) bacteriophage f2;
(j) bacteriophage PP7; and
(k) bacteriophage AP205.

38. The process of claim 27, wherein said RNA bacteriophage is bacteriophage Qβ.

39. The process of claim 27, wherein the purity of said composition is at least 99.5%, as determined by size exclusion chromatography.

40. A nucleotide composition comprising aggregated oligonucleotides,
wherein said nucleotide composition is obtainable by the process comprising steps of:
(a) providing oligonucleotides which comprise 3 to 15 guanosine entities at the 5′ end and 3 to 15 guanosine entities at the 3′ end, and wherein said oligonucleotides comprise phosphodiester connected deoxynucleotides;
(b) denaturing said oligonucleotides, wherein said denaturing comprises a step of incubating an aqueous solution I comprising said oligonucleotides and a chaotropic agent at 75° C. to 99° C. until the average diameter of said oligonucleotides is 1 nm or less, wherein said average diameter is determined by Dynamic Light Scattering (DLS), and
(c) aggregating said oligonucleotides, wherein said aggregating comprises the steps of
(i) incubating an aqueous solution II comprising said oligonucleotides having said average diameter of 1 nm or less obtained in step (b), a chaotropic agent and a cation at 75° C. to 99° C. to form said aggregated oligonucleotides, wherein said incubating is performed until the average diameter of said formed aggregated oligonucleotides is 6-16 nm, wherein said average diameter is determined by Dynamic Light Scattering (DLS), and
(ii) adjusting the temperature of said solution II to below 40° C.

41. The nucleotide composition of claim 39, wherein at least 90% said aggregated oligonucleotides have an average diameter of 7-14 nm, or
wherein at least 90% of said aggregated oligonucleotides have a diameter of 10.8 nm to 13.2 nm, or
wherein at least 65% of said aggregated oligonucleotides have a diameter of 11.4 nm to 12.6 nm,
wherein said average diameter is determined by Dynamic Light Scattering (DLS).

42. The nucleotide composition of claim 39, wherein said aggregated oligonucleotides have the nucleic acid sequence comprise a nucleotide sequence selected from the group consisting of:
(a) G10:
(SEQ ID NO: 1)
GGGGGGGGGGGACGATCGTCGGGGGGGGGG;
(b) G10-11:
(SEQ ID NO: 3)
GGGGGGGGGGGACGATCGTCGGGGGGGGGGG;
(c) G12-11:
(SEQ ID NO: 4)
GGGGGGGGGGGGGACGATCGTCGGGGGGGGGGG
(d) G6:
(SEQ ID NO: 5)
GGGGGGGACGATCGTCGGGGGG;
(e) G7:
(SEQ ID NO: 6)
GGGGGGGGACGATCGTCGGGGGGG;
(f) G8:
(SEQ ID NO: 7)
GGGGGGGGGACGATCGTCGGGGGGGG;
(g) G9:
(SEQ ID NO: 8)
GGGGGGGGGGACGATCGTCGGGGGGGGG;
(h) G11:
(SEQ ID NO: 9)
GGGGGGGGGGGGACGATCGTCGGGGGGGGGGG
(i) G6-10:
(SEQ ID NO: 24)
GGGGGGGACGATCGTCGGGGGGGGGG;
(j) G7-10:
(SEQ ID NO: 25)
GGGGGGGGACGATCGTCGGGGGGGGGG;
(k) G8-10:
(SEQ ID NO: 26)
GGGGGGGGGACGATCGTCGGGGGGGGGG;
and
(l) G9-10:
(SEQ ID NO: 27)
GGGGGGGGGGACGATCGTCGGGGGGGGGG.

43. A composition comprising
(1) a virus-like particle of an RNA bacteriophage, and
(2) aggregated oligonucleotides, wherein said aggregated oligonucleotides are packaged into said virus-like particle,
wherein the composition is obtained by a process comprising the steps of
(A) generating a mixture, wherein said mixture comprises:
(i) a coat protein of said RNA bacteriophage;
(ii) an agent capable of preventing the self-assembly of said coat protein; and
(iii) aggregated oligonucleotides, wherein said aggregated oligonucleotides comprise oligonucleotides comprising at least one poly G stretch;
(B) removing said agent from said mixture; and
(C) allowing said coat protein to self-assemble into a virus-like particle and to package said aggregated oligonucleotides;
wherein said aggregated oligonucleotides are obtained by a process comprising
(a) providing oligonucleotides which comprise 3 to 15 guanosine entities at the 5′ end and 3 to 15 guanosine entities at the 3′ end, and wherein said oligonucleotides comprise phosphodiester connected deoxynucleotides;
(b) denaturing said oligonucleotides, wherein said denaturing comprises the step of
(i) incubating an aqueous solution I comprising said oligonucleotides and a chaotropic agent at 75° C. to 99° C. until the average diameter of said oligonucleotides is 1 nm or less, wherein said average diameter is determined by Dynamic Light Scattering (DLS), and
(c) aggregating said oligonucleotides, wherein said aggregating comprises the steps of
(i) incubating an aqueous solution II comprising said oligonucleotides having said average diameter of 1 nm or less obtained in step (b), a chaotropic agent and a cation at 75° C. to 99° C. to form said aggregated oligonucleotides, wherein said incubating is performed until the average diameter of said formed aggregated oligonucleotides is 6-16 nm, wherein said average diameter is determined by Dynamic Light Scattering (DLS), and
(ii) adjusting the temperature of said solution II to below 40° C.

44. The composition of claim 43, wherein said aggregated oligonucleotides have the nucleic acid sequence comprise a nucleotide sequence selected from the group consisting of:
(a) G10:
(SEQ ID NO: 1)
GGGGGGGGGGGACGATCGTCGGGGGGGGGG;
(b) G10-11:
(SEQ ID NO: 3)
GGGGGGGGGGGACGATCGTCGGGGGGGGGGG;
(c) G12-11:
(SEQ ID NO: 4)
GGGGGGGGGGGGGACGATCGTCGGGGGGGGGGG
(d) G6:
(SEQ ID NO: 5)
GGGGGGGACGATCGTCGGGGGG;
(e) G7:
(SEQ ID NO: 6)
GGGGGGGGACGATCGTCGGGGGGG;
(f) G8:
(SEQ ID NO: 7)
GGGGGGGGGACGATCGTCGGGGGGGG;
(g) G9:
(SEQ ID NO: 8)
GGGGGGGGGGACGATCGTCGGGGGGGGG;
(h) G11:
(SEQ ID NO: 9)
GGGGGGGGGGGGACGATCGTCGGGGGGGGGGG
(i) G6-10:
(SEQ ID NO: 24)
GGGGGGGACGATCGTCGGGGGGGGGG;
(j) G7-10:
(SEQ ID NO: 25)
GGGGGGGGACGATCGTCGGGGGGGGGG;
(k) G8-10:
(SEQ ID NO: 26)
GGGGGGGGGACGATCGTCGGGGGGGGGG;
and
(l) G9-10:
(SEQ ID NO: 27)
GGGGGGGGGGACGATCGTCGGGGGGGGGG.

45. The composition of claim 43, wherein said RNA bacteriophage is selected from the group consisting of:
(a) bacteriophage Qβ;
(b) bacteriophage R17;
(c) bacteriophage fr;
(d) bacteriophage GA;
(d) bacteriophage SP;
(e) bacteriophage MS2;
(f) bacteriophage M11;
(g) bacteriophage MX1;
(h) bacteriophage NL95;
(i) bacteriophage f2;
(j) bacteriophage PP7; and
(k) bacteriophage AP205.

46. The composition of claim 43, wherein the purity of said composition comprising the virus-like particle and aggregated oligonucleotides is at least 99.5% as determined by size exclusion chromatography.
